(19) United States
(12) Patent Application Publication
(10) Pub. No.: US 2011/0159017 A1
(43) Pub. Date: Jun. 30, 2011

(54) TRYTOPHAN CATABOLISM IN CANCER TREATMENT AND DIAGNOSIS

(75) Inventors: Benoit Van Den Eynde, Brussels (BE); Luc Pilotte, Brussels (BE); Etienne De Plaen, Brussels (BE)

(73) Assignee: Ludwig Institute for Cancer Research Ltd., New York, NY (US)

(21) Appl. No.: 12/936,576

(22) PCT Filed: Apr. 10, 2009
(86) PCT No.: PCT/US09/02250

§ 371 Date: Mar. 2, 2011

Related U.S. Application Data
(63) Continuation of application No. 61/123,940, filed on Apr. 11, 2008.

(51) Int. Cl. A61K 39/395; C12Q 1/68; G01N 33/574; A61K 31/4439; A61K 31/192; A61K 31/40; A61K 31/225; A61K 31/198; A61K 35/12; A61K 31/353; A61K 31/405; C12N 5/09; C12N 5/0783; A61P 35/00

(52) U.S. Cl. 424/174.1; 435/6.12; 435/6.14; 435/7.1; 435/7.92; 514/339; 514/569; 514/423; 514/547; 514/562; 424/93.71; 514/548; 514/456; 514/419; 435/375

(57) ABSTRACT

The unexpected expression of tryptophan 2,3-dioxygenase (TDO2) in cancer cells and tumors has been established. Methods for diagnosing cancer based on the expression of TDO2 are provided, as are methods for treating cancer and inhibiting the growth of cancer cells by inhibiting TDO2, as well as pharmaceutical compositions.

### RELATED APPLICATION

This application claims the benefit under 35 USC 119 of U.S. provisional application Ser. No. 61/123,940, filed Apr. 11, 2008, the entire disclosure of which is incorporated by reference herein.

### BACKGROUND OF THE INVENTION

Cancers and tumors regularly evade the immune surveillance system of the host employing several mechanisms. It was found previously that one mechanism resulting in tumoral immune resistance stems from the constitutive expression of indoleamine 2,3-dioxygenase (IDO) by tumor cells (Uyttenhove, C., L. Pilotte, I. Theate, V. Stroobant, D. Colau, N. Parmentier, T. Boon, and B. J. Van den Eynde. 2003. Nat. Med. 9:1269-1274). IDO degrades tryptophan into kynurenine intracellularly, inducing a drop in the intracellular concentration of tryptophan. Tryptophan then passively enters the cell through a transporter of the L-system of amino acid transporters, according to the tryptophan concentration gradient. The resulting local drop in extracellular tryptophan in the vicinity of IDO-expressing cells is deleterious to T lymphocyte survival and proliferation in the microenvironment. While tumor cells expressing high levels of IDO have a reduced rate of growth in vitro, their proliferation is not arrested. T lymphocytes, in contrast, stop proliferating under such conditions because they have a tryptophan-sensitive checkpoint. IDO is not expressed in normal tissues except in the placenta, where it appears to contribute to immune tolerance of the fetus by the maternal immune system (Munn, D. H., M. Zhou, J. T. Attwood, I. Bondarev, S. J. Conway, B. Marshall, C. Brown, and A. L. Mellor. 1998. Science 281:1191-1193). IDO is also inducible with interferon-gamma, an inflammatory cytokine. Most tumors constitutively express IDO, and IDO expression renders these tumor cells resistant to immune rejection. It was found that pharmacological inhibition of IDO, using specific inhibitors, promotes immune rejection of IDO-expressing tumors (Uyttenhove, C., L. Pilotte, I. Theate, V. Stroobant, D. Colau, N. Parmentier, T. Boon, and B. J. Van den Eynde. 2003. Nat. Med. 9:1269-1274, and references therein).

### SUMMARY OF THE INVENTION

It has now been discovered that a second key enzyme in tryptophan catabolism, tryptophan 2,3-dioxygenase (TDO2), unexpectedly is expressed in many tumor cells. Although TDO2 has no sequence similarity with IDO it also catalyzes the degradation of tryptophan into kynurenine. TDO2 expression in tumor cells appears to have an effect similar to expression of IDO, in that TDO2 expressed in tumors prevents tumor surveillance by the immune system and thus prevents tumor rejection by locally degrading tryptophan.

According to one aspect of the invention, methods for diagnosing cancer in a subject are provided. The methods include determining the expression of tryptophan 2,3-dioxygenase (TDO2) in a subject. The expression of TDO2 is determined in a subject by obtaining a sample containing cells from the subject that is not a liver sample, and measuring the expression of TDO2 in the sample. The expression of TDO2 in the sample indicates that the subject has cancer or is at risk for having cancer. In some embodiments, the sample containing cells from the subject is not a skin or bladder sample.

In some embodiments, the determination of TDO2 expression is carried out measuring TDO2 mRNA or protein in the sample. In certain embodiments, the TDO2 mRNA in the sample is measured by PCR. For example, in some embodiments the PCR is real time RT-PCR, including quantitative RT-PCR. In other embodiments, the TDO2 protein in the sample is measured by an immunoassay using an antibody that specifically binds TDO2 protein. In certain embodiments, the immunoassay is an ELISA assay.

In some embodiments, the method comprises contacting the sample with one or more reagents for measuring expression of TDO2. In certain embodiments, the reagents are contacted with the sample under conditions that cause formation of an amplification product of TDO2 RNA. In other embodiments, the reagents are contacted with the sample under conditions that cause formation of a complex of at least one of the reagents and TDO2 protein.

In certain embodiments, the subject is diagnosed as having cancer or as being at risk for having cancer if the expression of TDO2 in the sample is more than 0.3 TDO2 molecules per cell, more than 0.4 TDO2 molecules per cell, more than 0.5 TDO2 molecules per cell, more than 0.6 TDO2 molecules per cell, more than 0.7 TDO2 molecules per cell, more than 0.8 TDO2 molecules per cell, more than 0.9 TDO2 molecules per cell, more than 1.0 TDO2 molecules per cell, more than 1.1 TDO2 molecules per cell, more than 1.2 TDO2 molecules per cell, more than 1.3 TDO2 molecules per cell, more than 1.4 TDO2 molecules per cell, more than 1.5 TDO2 molecules per cell, more than 1.6 TDO2 molecules per cell, more than 1.7 TDO2 molecules per cell, more than 1.8 TDO2 molecules per cell, more than 1.9 TDO2 molecules per cell, more than 2.0 TDO2 molecules per cell, more than 2.1 TDO2 molecules per cell, more than 2.2 TDO2 molecules per cell, more than 2.3 TDO2 molecules per cell, more than 2.4 TDO2 molecules per cell, more than 2.5 TDO2 molecules per cell, more than 2.6 TDO2 molecules per cell, more than 2.7 TDO2 molecules per cell, more than 2.8 TDO2 molecules per cell, more than 2.9 TDO2 molecules per cell, more than 3.0 TDO2 molecules per cell, more than 3.1 TDO2 molecules per cell, more than 3.2 TDO2 molecules per cell, more than 3.3 TDO2 molecules per cell, more than 3.4 TDO2 molecules per cell, more than 3.5 TDO2 molecules per cell, more than 3.6 TDO2 molecules per cell, more than 3.7 TDO2 molecules per cell, more than 3.8 TDO2 molecules per cell, more than 3.9 TDO2 molecules per cell, more than 4 TDO2 molecules per cell, more than 5 TDO2 molecules per cell, more than 6 TDO2 molecules per cell, more than 7 TDO2 molecules per cell, more than 8 TDO2 molecules per cell, more than 9 TDO2 molecules per cell, more than 10 TDO2 molecules per cell, more than 11 TDO2 molecules per cell, more than 12 TDO2 molecules per cell, more than 13 TDO2 molecules per cell, more than 14 TDO2 molecules per cell, more than 15 TDO2 molecules per cell, more than 16 TDO2 molecules per cell, more than 17 TDO2 molecules per cell, more than 18 TDO2 molecules per cell, more than 19 TDO2 molecules per cell, more than 20 TDO2 molecules per cell, or more than 30, 40, 50, 60, 70, 80, 90, or 100 TDO2 molecules per cell.

Kits for practicing the foregoing diagnostic methods also are provided.

According to another aspect of the invention, methods are provided for treating a subject having or suspected of having a cancer, or being at risk of developing a cancer. The methods include administering to the subject in need of such treatment an amount of an inhibitor effective to inhibit the activity of tryptophan 2,3-dioxygenase (TDO2). In certain embodiments, the cells of the cancer express tryptophan 2,3-dioxygenase (TDO2). In some embodiments the TDO2 inhibitor is selected from the group consisting of 680C91 ((E)-6-fluoro-3-[2-(3-pyridyl)vinyl]-1H-indole), 709W92 ((E)-6-fluoro-3-[2-(4-pyridyl)vinyl]-1H-indole), Sulindac (2-[6-fluoro-2-methyl-3-[(4-methylsulfinylphenyl)methylidene]inden-1-yl]-acetic acid), and 540C91 ((E)-3-[2-(4′-pyridyl)-vinyl]-1H-indole), Tolmetin (2-[1-methyl-5-(4-methylbenzoyl)-pyrrol-2-yl]acetic acid).

In certain embodiments, the TDO2 inhibitor is a heme depleting agent and the heme depleting agent reduces the availability of heme to the TDO2 polypeptide. In some embodiments, the heme depleting agent is an agent that induces the expression of heme oxygenase-1 (HSP32). In certain embodiments the heme oxygenase-1 inducing agent is a glutathione-reducing agent, preferably diethyl maleate (DEM) or L-buthionine-(S,R)-sulfoximine (BSO).

In certain embodiments, the TDO2 inhibitor is a siRNA specific for a TDO2 gene transcript, and the siRNA reduces the amount of TDO2 mRNA and TDO2 protein in the TDO2 expressing cell. In some embodiments the siRNA nucleic acid molecule includes one or more modified nucleotides or nucleosides that enhance in vivo stability. In certain embodiments the siRNA nucleic acid molecule includes one or more modified nucleotides or nucleosides that enhance transport across a cell membrane.

In certain embodiments, the TDO2 inhibitor is an antibody, or antigen-binding fragment thereof, wherein the antibody, or antigen-binding fragment thereof, specifically binds to the TDO2 polypeptide. Binding of the antibody, or antigen-binding fragment thereof, to the TDO2 polypeptide reduces the catalytic activity of the TDO2 polypeptide; the catalytic activity of the TDO2 polypeptide is degradation of tryptophan. In certain embodiments the antibody is a monoclonal antibody, a human antibody, a domain antibody, a humanized antibody, a single chain antibody or a chimeric antibody. In certain embodiments the antibody fragment is a F(ab′)₂, Fab, Fd, or Fv fragment. In some embodiments the antibody is polyclonal.

In certain embodiments, the TDO2 inhibitor is a nucleic acid molecule including an antisense sequence that hybridizes to TDO2 gene or mRNA; hybridization of the antisense sequence to TDO2 gene reduces the amount of RNA transcribed from the TDO2 gene. In some embodiments hybridization of the antisense sequence to the TDO2 mRNA reduces the amount of protein translated from TDO2 mRNA, and/or alters the splicing of TDO2 mRNA. In certain embodiments the nucleic acid molecule includes one or more modified nucleotides or nucleosides that enhance in vivo stability, transport across a cell membrane, or hybridization to TDO2 gene or mRNA.

According to another aspect of the invention, any of the aforementioned methods further include administering to a subject an amount of an indoleamine 2,3-dioxygenase (IDO) inhibitor effective to inhibit IDO. In certain embodiments the IDO inhibitor is selected from the group consisting of 1-methyl-DL-tryptophan, 1-methyl-L-tryptophan, β-(3-benzofuranyl)-DL-alanine, β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, 5-bromoindoxyl diacetate, 5-bromo-4-chloroindoxyl 1,3-diacetate, annulin A, annulin B, annulin C, Brassinin derivatives, necrostatin 1/methylthiohydantoine-tryptophan(5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan), naphtoquinones, p-Coumarinic acid((E)-3-(4-Hydroxyphenyl)-2-propenoic acid), Rosmarinic acid((2R)-2-[[(2E)-3-(3,4-Dihydroxyphenyl)-1-oxo-2-propenyl]]oxy]-3-(3,4-dihydroxyphenyl)propanoic acid), and Epigallocatechin.

According to another aspect of the invention, any of the aforementioned methods further include administering one or more anti-cancer agents. In certain embodiments the anti-cancer agent is selected from the group consisting of cytotoxic T cells (CTL), cytotoxic antibodies, cytotoxic or growth-inhibitory chemotherapeutic agents, and anti-vasculature or anti-angiogenesis agents.

According to yet another aspect of the invention, any of the aforementioned methods further include administering one or more immune modulators. In certain embodiments the immune modulator is selected from the group consisting of antigen-specific T lymphocytes, peptide antigens, antigenic proteins and nucleic acids. In certain embodiments the immune modulator further comprises an adjuvant. In certain embodiments the adjuvant is selected from the group consisting of monophosphoryl lipid A (MPL); saponins including QS21, DQS21, QS-7, QS-17, QS-18, and QS-L1; DQS21/MPL; CpG; montanide; and water-in-oil emulsions prepared from biodegradable oils.

In some embodiments of the aforementioned methods, expression of tryptophan 2,3-dioxygenase (TDO2) is determined in a subject prior to treatment comprising administering to the subject an amount of an inhibitor effective to inhibit the activity of tryptophan 2,3-dioxygenase (TDO2). The expression of TDO2 is determined by obtaining a sample containing cells from the subject, that is not a liver sample, and measuring the expression of TDO2 in the sample. In certain embodiments the methods further include determining the expression of indoleamine 2,3-dioxygenase (IDO) in the subject. In certain embodiments the expression of TDO2 or TDO2 and IDO is determined by measuring TDO2 or TDO2 and IDO mRNA or protein in the sample. In certain embodiments, the sample is not a skin or bladder sample.

According to yet another aspect of the invention, methods for inhibiting the growth or killing cancer cells which have evaded or have the potential to evade T cell-mediated cytolysis are provided. The methods include contacting the cancer cells with an amount of an inhibitor of tryptophan 2,3-dioxygenase (TDO2) effective to increase T cell-mediated cytolysis of the cancer cells, wherein the cancer cells express TDO2, thereby inhibiting the growth or killing the cancer cells.

In certain embodiments, the cancer cells express tryptophan 2,3-dioxygenase (TDO2). In certain embodiments the TDO2 inhibitor is selected from the group consisting of 680C91 ((E)-6-fluoro-3-[2-(3-pyridyl)vinyl]-1H-indole), 709W92 ((E)-6-fluoro-3-[2-(4-pyridyl)vinyl]-1H-indole), Tolmetin (2-[1-methyl-5-(4-methylbenzoyl)-pyrrol-2-yl]acetic acid), and Sulindac (2-[6-fluoro-2-methyl-3-[(4-methylsulfinylphenyl)methylidene]inden-1-yl)-acetic acid), and 540C91 ((E)-3-[2-(4′-pyridyl)-vinyl]-1H-indole).

In some embodiments, the TDO2 inhibitor is a heme depleting agent, wherein the heme depleting agent reduces the availability of heme to the TDO2 polypeptide. In certain embodiments the heme depleting agent is an agent that induces the expression of heme oxygenase-1 (HSP32). In some embodiments the heme oxygenase-1 inducing agent is a glutathione-reducing agent, preferably diethyl maleate (DEM) or L-buthionine-(S,R)-sulfoximine (BSO).

In some embodiments, the TDO2 inhibitor is a siRNA specific for a TDO2 gene transcript, and wherein the siRNA reduces the amount of TDO2 mRNA and TDO2 protein in the TDO2 expressing cell. In certain embodiments, the siRNA nucleic acid molecule includes one or more modified nucleotides or nucleosides that enhance in vivo stability. In certain embodiments the siRNA nucleic acid molecule includes one or more modified nucleotides or nucleosides that enhance transport across a cell membrane.

In some embodiments, the TDO2 inhibitor is an antibody, or antigen-binding fragment thereof, wherein the antibody, or antigen-binding fragment thereof, specifically binds to the TDO2 polypeptide, and wherein binding of the antibody, or antigen-binding fragment thereof, to the TDO2 polypeptide reduces the catalytic activity of the TDO2 polypeptide; the catalytic activity of the TDO2 polypeptide is degradation of tryptophan. In certain embodiments the antibody is a monoclonal antibody, a human antibody, a domain antibody, a humanized antibody, a single chain antibody or a chimeric antibody. In certain embodiments the antibody fragment is a F(ab′)₂, Fab, Fd, or Fv fragment. In certain embodiments the antibody is polyclonal.

In some embodiments, the TDO2 inhibitor is a nucleic acid molecule comprising an antisense sequence that hybridizes to TDO2 gene or mRNA; hybridization of the antisense sequence to TDO2 gene reduces the amount of RNA transcribed from the TDO2 gene. In certain embodiments, hybridization of the antisense sequence to the TDO2 mRNA reduces the amount of protein translated from TDO2 mRNA, and/or alters the splicing of TDO2 mRNA. In some embodiments the nucleic acid molecule includes one or more modified nucleotides or nucleosides that enhance in vivo stability, transport across a cell membrane, or hybridization to TDO2 gene or mRNA.

According to another aspect of the invention, any of the aforementioned methods for inhibiting the growth or killing cancer cells which have evaded or have the potential to evade T cell-mediated cytolysis further include contacting the cancer cells with an amount of an indoleamine 2,3-dioxygenase (IDO) inhibitor effective to inhibit IDO. In certain embodiments, the IDO inhibitor is selected from the group consisting of 1-methyl-DL-tryptophan, 1-methyl-L-tryptophan, β-(3-benzofuranyl)-DL-alanine, β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, and 5-bromoindoxyl diacetate, and 5-bromo-4-chloroindoxyl 1,3-diacetate, annulin A, annulin B, annulin C, Brassinin derivatives, necrostatin 1/methylthiohydantoine-tryptophan (5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan), naphtoquinones, p-Coumarinic acid((E)-3-(4-Hydroxyphenyl)-2-propenoic acid), Rosmarinic acid((2R)-2-[[(2E)-3-(3,4-Dihydroxyphenyl)-1-oxo-2-propenyl]]oxy]-3-(3,4-dihydroxyphenyl)propanoic acid), and Epigallocatechin.

According to another aspect of the invention, any of the aforementioned methods for inhibiting the growth or killing cancer cells which have evaded or have the potential to evade T cell-mediated cytolysis further include contacting the cancer cells with one or more anti-cancer agents. In certain embodiments, the anti-cancer agent is selected from the group consisting of cytotoxic T cells (CTL), cytotoxic antibodies, cytotoxic or growth-inhibitory chemotherapeutic agents, and anti-vasculature or anti-angiogenesis agents.

According to another aspect of the invention, any of the aforementioned methods for inhibiting the growth or killing cancer cells which have evaded or have the potential to evade T cell-mediated cytolysis further include contacting the cell with one or more immune modulators. In certain embodiments the immune modulator is selected from the group consisting of antigen-specific T lymphocytes, peptide antigens, antigenic proteins and nucleic acids. In some embodiments the immune modulator further comprises an adjuvant. In certain embodiments the adjuvant is selected from the group consisting of monophosphoryl lipid A (MPL); saponins including QS21, DQS21, QS-7, QS-17, QS-18, and QS-L1; DQS21/MPL; CpG; montanide; and water-in-oil emulsions prepared from biodegradable oils.

According to yet another aspect of the invention, pharmaceutical compositions are provided. The pharmaceutical compositions include an amount of a tryptophan 2,3-dioxygenase (TDO2) inhibitor effective to inhibit TDO2 and increase local tryptophan concentrations in the presence of TDO2 polypeptide expression, and a pharmaceutically acceptable carrier. In certain embodiments the TDO2 inhibitor is selected from the group consisting of 680C91 ((E)-6-fluoro-3-[2-(3-pyridyl)vinyl]-1H-indole), 709W92 ((E)-6-fluoro-3-[2-(4-pyridyl)vinyl]-1H-indole), Tolmetin (2-[1-methyl-5-(4-methylbenzoyl)-pyrrol-2-yl]acetic acid), and Sulindac (2-[6-fluoro-2-methyl-3-[(4-methylsulfinylphenyl)methylidene]inden-1-yl]-acetic acid), and 540C91 ((E)-3-[2-(4′-pyridyl)-vinyl]-1H-indole).

In some embodiments, the TDO2 inhibitor is a heme depleting agent, wherein the heme depleting agent reduces the availability of heme to the TDO2 polypeptide. In certain embodiments the heme depleting agent is an agent that induces the expression of heme oxygenase-1 (HSP32). In certain embodiments the heme oxygenase-1 inducing agent is a glutathione-reducing agent, preferably diethyl maleate (DEM) or L-buthionine-(S,R)-sulfoximine (BSO).

In some embodiments, the TDO2 inhibitor is a siRNA specific for a TDO2 gene transcript, and wherein the siRNA reduces the amount of TDO2 mRNA and TDO2 protein in the TDO2 expressing cell. In certain embodiments the siRNA nucleic acid molecule includes one or more modified nucleotides or nucleosides that enhance in vivo stability. In some embodiments the siRNA nucleic acid molecule includes one or more modified nucleotides or nucleosides that enhance transport across a cell membrane.

In some embodiments, the TDO2 inhibitor is an antibody, or antigen-binding fragment thereof, wherein the antibody, or antigen-binding fragment thereof, specifically binds to the TDO2 polypeptide, and binding of the antibody, or antigen-binding fragment thereof, to the TDO2 polypeptide reduces the catalytic activity of the TDO2 polypeptide, wherein the catalytic activity of the TDO2 polypeptide is degradation of tryptophan. In certain embodiments the antibody is a monoclonal antibody, a human antibody, a domain antibody, a humanized antibody, a single chain antibody or a chimeric antibody. In certain embodiments the antibody fragment is a F(ab′)₂, Fab, Fd, or Fv fragment. In certain embodiments the antibody is polyclonal.

In some embodiments, the TDO2 inhibitor is a nucleic acid molecule comprising an antisense sequence that hybridizes to TDO2 gene or mRNA; and wherein hybridization of the antisense sequence to TDO2 gene reduces the amount of RNA transcribed from the TDO2 gene. In certain embodiments hybridization of the antisense sequence to the TDO2 mRNA reduces the amount of protein translated from TDO2 mRNA, and/or alters the splicing of TDO2 mRNA. In certain embodiments the nucleic acid molecule includes one or more modified nucleotides or nucleosides that enhance in vivo stability, transport across a cell membrane, or hybridization to TDO2 gene or mRNA.

According to yet another aspect of the invention, any of the aforementioned pharmaceutical compositions further include an indoleamine 2,3-dioxygenase (IDO) inhibitor effective to inhibit IDO and to increase local tryptophan concentrations in the presence of TDO2 polypeptide expression. In certain embodiments the IDO inhibitor is selected from the group consisting of 1-methyl-DL-tryptophan, β-(3-benzofuranyl)-DL-alanine, β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, and 5-bromoindoxyl diacetate, and 5-bromo-4-chloroindoxyl 1,3-diacetate annulin A, annulin B, annulin C, Brassinin derivatives, necrostatin 1/methylthiohydantoine-tryptophan(5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan), naphtoquinones, p-Coumarinic acid((E)-3-(4-Hydroxyphenyl)-2-propenoic acid), Rosmarinic acid((2R)-2-[[(2E)-3-(3,4-Dihydroxyphenyl)-1-oxo-2-propenyl]]oxy]-3-(3,4-dihydroxyphenyl)propanoic acid), and Epigallocatechin.

According to yet another aspect of the invention, any of the aforementioned pharmaceutical compositions further include one or more anti-cancer agents. In certain embodiments the anti-cancer agent is selected from the group consisting of cytotoxic T cells (CTL), cytotoxic antibodies, cytotoxic or growth-inhibitory chemotherapeutic agents, and anti-vasculature or anti-angiogenesis agents.

According to yet another aspect of the invention, any of the aforementioned pharmaceutical compositions further include one or more immune modulators. In certain embodiments the immune modulator is selected from the group consisting of antigen-specific T lymphocytes, peptide antigens, antigenic proteins and nucleic acids. In some embodiments the pharmaceutical composition further comprises an adjuvant. In certain embodiments the adjuvant is selected from the group consisting of monophosphoryl lipid A (MPL); saponins including QS21, DQS21, QS-7, QS-17, QS-18, and QS-Ll; DQS21/MPL; CpG; montanide; and water-in-oil emulsions prepared from biodegradable oils.

According to another aspect of the invention, methods for increasing proliferation of T lymphocytes are provided. The methods include growing the T lymphocytes by culturing in vitro in the presence of an amount of one or more inhibitors of tryptophan 2,3-dioxygenase (TDO2) effective to increase the proliferation of the T lymphocytes at least about 10% relative to a control population of T lymphocytes that is cultured without the one or more TDO2 inhibitors. In certain embodiments, the TDO2 inhibitor is selected from the group consisting of 680C91 ((E)-6-fluoro-3-[2-(3-pyridyl)vinyl]-1H-indole), 709W92 ((E)-6-fluoro-3-[2-(4-pyridyl)vinyl]-1H-indole), Tolmetin (2-[1-methyl-5-(4-methylbenzoyl)-pyrrol-2-yl]acetic acid), and Sulindac (2-[6-fluoro-2-methyl-3-[(4-methylsulfinylphenyl)methylidene]inden-1-yl]-acetic acid), and 540C91 ((E)-3-[2-(4′-pyridyl)-vinyl]-1H-indole).

In certain embodiments the methods further include growing the cells in the presence of an additional tryptophan enhancing agent. In certain embodiments, the additional tryptophan enhancing agent is an inhibitor of indoleamine 2,3-dioxygenase (IDO). In some embodiments, the IDO inhibitor is selected from the group consisting of 1-methyl-DL-tryptophan, 1-methyl-L-tryptophan, β-(3-benzofuranyl)-DL-alanine and β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, and 5-bromoindoxyl diacetate, and 5-bromo-4-chloroindoxyl 1,3-diacetate annulin A, annulin B, annulin C, Brassinin derivatives, necrostatin 1/methylthiohydantoine-tryptophan(5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan), naphtoquinones, p-Coumarinic acid((E)-3-(4-Hydroxyphenyl)-2-propenoic acid), Rosmarinic acid((2R)-2-[[(2E)-3-(3,4-Dihydroxyphenyl)-1-oxo-2-propenyl]]oxy]-3-(3,4-dihydroxyphenyl)propanoic acid), and Epigallocatechin.

In some embodiments, proliferation of the T lymphocytes is increased at least about 50% relative to a control population of T lymphocytes that is cultured without TDO2 inhibitor.

In some embodiments, the T lymphocytes are taken from a host and are cultured ex vivo with cancer cells from the host to increase specificity of cytolytic activity of the T lymphocytes relative to a control population of T lymphocytes that is cultured without TDO2 inhibitor. In certain embodiments the specific cytolytic T lymphocytes are reintroduced into the host.

The compositions provided herein include an amount of a tryptophan enhancing agent effective to increase local tryptophan concentrations in the presence of TDO2 or TDO2 and IDO expression. The composition also includes a pharmaceutically acceptable carrier. In other aspects of the invention, methods for treating cancer cells which have evaded or have the potential to evade T cell-mediated cytolysis are provided. The methods include administering to a subject in need of such treatment an amount of a tryptophan enhancing agent effective to increase T cell-mediated cytolysis of the cancer cell. In certain embodiments, the cancer cells expresses TDO2 or both TDO2 and IDO. Other methods include contacting the cancer cells with an amount of a tryptophan enhancing agent effective to increase T cell-mediated cytolysis of the cancer cell. Preferred tryptophan enhancing agents for use in these methods are set forth above.

The invention also provides pharmaceutical preparations containing any one or more of the compositions described herein. Such pharmaceutical preparations can include pharmaceutically acceptable diluents, carriers or excipients. The use of such compositions in the preparation of medicaments, particularly medicaments for the treatment of cancer and for increasing T cell proliferation also is provided.

These and other aspects of the invention, as well as various advantages and utilities will be more apparent with reference to the drawings and detailed description of the invention.

### BRIEF DESCRIPTION OF THE FIGURES

FIG. 1. A photograph of a gel loaded with RT-PCR amplified samples from normal tissue and cancer cell lines, detecting the expression of TDO2.

FIG. 2. A bar graph depicting tryptophan degradation by P815-mTDO transfected clones. 5×10⁵ cells of the indicated clone were incubated in 200 μl Hanks balanced salt solution (HBSS) containing 25 μM tryptophan. Tryptophan (light) and kynurenine (dark) concentrations (μM) were measured by HPLC in the supernatant after 4 hours at 37° C.

FIG. 3. Line graphs depicting tumor progression in mice injected with TDO-expressing cells. FIG. 3A shows clone 2; FIG. 3B shows clone 8.

FIG. 4. Line graphs depicting concentrations of tryptophan and kynurenine in culture supernatants of different transfected cells. Control human 293-EBNA cells (293-E) (FIG. 4A), 293-E cells transfected with human IDO (293-E hIDO) (FIG. 4B) or human 293-E cells transfected with human TDO2 (293-E hTDO) (FIG. 4C) were incubated overnight (4×10⁵ cells/2000) in Hank's balanced Salt solution (HBSS) supplemented with 25 μM tryptophan and increasing concentrations of the 680C91 inhibitor compound ([inh]). The concentrations (μM) of tryptophan ([Trp], ⋄) and kynurenin ([Kyn], □) were measured by HPLC in the supernatant.

FIG. 5. A bar graph depicting tryptophan consumption and kynurenine production in head and neck carcinoma cell line 2720 (SENY) in the presence or absence of the TDO-specific inhibitor 680C91 or the IDO inhibitor mb.

### DETAILED DESCRIPTION OF THE INVENTION

In humans the only source of tryptophan is the diet. Two critical enzymes are responsible for tryptophan catabolism, tryptophan 2,3-dioxygenase (TDO2, EC 1.13.1.12) and indoleamine 2,3-dioxygenase (IDO, EC 1.13.11.17), both catalyzing the degradation of tryptophan (Uyttenhove, C., L. Pilotte, I. Theate, V. Stroobant, D. Colau, N. Parmentier, T. Boon, and B. J. Van den Eynde. 2003. Nat. Med. 9:1269-1274; Comings, D. E., D. Muhleman, G. Dietz, M. Sherman, and G. L. Forest. 1995. Genomics 29:390-396; Salter, M., R. Hazelwood, C. I. Pogson, R. Iyer, and D. J. Madge. 1995. Biochem. Pharmacol. 49:1435-1442). The two enzymes share no apparent homology. TDO2 catalyzes the conversion of tryptophan to N-formyl L-kynurenine. It contains two heme and two copper molecules, both of which are essential for its catalytic activity. In the presence of tryptophan an apoenzyme-tryptophan complex forms that has a higher affinity for heme than the apoenzyme alone, and the complex competes more successfully with other intracellular heme-binding proteins for heme in the liver. TDO2 has a very rapid turnover rate in the liver. Association with tryptophan stabilizes the enzyme, thus preventing its rapid breakdown (Comings, D. E., D. Muhleman, G. Dietz, M. Sherman, and G. L. Forest. 1995. Genomics 29:390-396, and references therein). TDO2 is known to be expressed at high level in the liver, and its main function is to regulate systemic tryptophan levels by degrading excess dietary tryptophan.

It has now been discovered that tryptophan 2,3-dioxygenase (TDO2), unexpectedly, is expressed in many tumor cells. TDO2 expression in these tumor cells appears to have an effect similar to expression of IDO. TDO2 expressed in tumors prevents tumor surveillance by the immune system and thus prevents tumor rejection by locally degrading tryptophan.

Cancers and tumors regularly evade the immune surveillance system of the host employing several mechanisms. One such mechanism is the constitutive expression of IDO by tumor cells which leads to degradation of tryptophan and in turn results in a local drop in extracellular tryptophan in the vicinity of IDO-expressing cells, which is deleterious to T lymphocyte survival and proliferation in the microenvironment. TDO2 has now been shown to have similar effects (see Examples).

The molecular definition of human cancer antigens recognized by T cells has allowed the design of cancer immunotherapy protocols, based, for example, on vaccination with various antigen formulations. Expression of either TDO2 or both TDO2 and IDO can promote tumor resistance to therapy, such as, for example, vaccine-induced killing by the host immune system. This resistance can be overcome, for example by systemic inhibition of either TDO2 or both TDO2 and IDO by administering specific inhibitors of these enzymes, in subjects that would benefit from such treatment, such as patients undergoing immunotherapy.

To enhance immune surveillance and killing by T lymphocytes in certain embodiments the invention provides compounds that are tryptophan enhancing agents, which are in some embodiments inhibitors of TDO2 or both TDO2 and IDO, and which can be administered in a prophylactic or therapeutic manner.

Cancer cells that express TDO2 or both TDO2 and IDO would be expected to reduce the local concentration of tryptophan and disable T cell-mediated immune responses to the cancer. The recognition of this unexpected property of cancer cells permits treatment of the cells, which may have evaded or have the potential to evade T cell-mediated cytolysis, to increase immune recognition and destruction of the cancer cells. Certain embodiments of the invention include administering to a person having cancer, or being suspected of having cancer, or being at risk of developing a cancer, therapeutically or prophylactically an amount of a tryptophan enhancing agent effective to increase T cell-mediated cytolysis of the cancer cell. In certain embodiments methods of the invention include contacting the cancer cells with an amount of a tryptophan enhancing agent effective to increase T cell-mediated cytolysis of the cancer cell. Preferred tryptophan enhancing agents are described elsewhere herein. TDO2 or both TDO2 and IDO inhibitors can be administered to any person having cancer, or being suspected of having cancer, or being at risk of developing a cancer, alone or in combination with other cancer treatment regimens, described elsewhere herein, whether or not the expression status of TDO2 or both TDO2 and IDO in the cancer cells is known; it is sufficient to determine or suspect that a person has cancer.

The recognition that cancer cells'can express TDO2 or both TDO2 and IDO, also permits one of ordinary skill in the art to determine a condition characterized by the ability of cancer cells to resist or evade T cell-mediated cytolysis. For example, one can monitor a sample of cells, preferably not derived from the liver, and in some embodiments is not derived from the skin or bladder, from a patient who has or is suspected of having cancer, or is at risk of developing a cancer, for expression of TDO2 or both TDO2 and IDO, as a determination of the condition. Once it is established that the subject has a cancer that expresses TDO2 or both TDO2 and IDO, the skilled artisan can determine whether to treat the cancer patient with an inhibitor of TDO2 or both TDO2 and IDO, such as provided herein. Expression of TDO2 or both TDO2 and IDO by the cancer cells indicates that the patient is a candidate to be treated with an inhibitor of TDO2 or both TDO2 and IDO to increase the susceptibility of the cancer cells to T cell attack. In these methods, expression of TDO2 or both TDO2 and IDO by the cancer cells of the patient can be monitored by any method, including measuring the amount of TDO2 protein or IDO protein, or both or nucleic acids encoding the enzymes that are expressed by the cancer cells. TDO2 and/or IDO proteins can be measured directly, such as by standard immunoassays, or indirectly, such as by measuring TDO2 and/or IDO enzymatic activity in accordance with known methods. TDO2 and/or IDO nucleic acids can be measured by nucleic acid hybridization or amplification, such as PCR.

In other embodiments the invention provides diagnostic tests to determine whether a subject has cancer or is at risk of developing cancer, wherein a cell sample is taken from the subject, preferably not derived from the liver, and in some embodiments not derived from the skin or bladder, and subjected to a measurement of a determinant that is specific for cancer, such as a quantitative measurement. In some embodiments the determinant is the quantity of total TDO2 mRNA (messenger RNA) from the sample or the number of mRNA molecules per cell in the sample. In other embodiments the determinant is the quantity of TDO2 protein in the sample, which can be measured for example by measuring the amount of protein per se or by measuring the enzymatic activity of TDO2 in the sample. If the quantity of TDO2 mRNA or protein is elevated in a non-liver (and optionally non-skin or non-bladder) cell sample of a subject compared to that of a control sample obtained from a subject not suffering from a cancer, then the subject is diagnosed to have a cancer or being at risk of developing a cancer. The methods can include contacting the sample with one or more reagents for measuring expression of TDO2. The methods may include isolation of nucleic acids or proteins from the sample, or the methods may be performed on the sample as obtained without isolation of nucleic acids or proteins from the sample.

TDO2 mRNA can be isolated from the cells isolated from the sample, which can be taken by any means known in the art, and the mRNA can be detected and quantified by any means known in the art, for example by RT-PCR (Reverse transcriptase-PCR analysis of mRNA), using reverse transcriptase to convert mRNA into complementary DNA (cDNA) which is then amplified by PCR, or preferably using real-time PCR (e.g., qRT-PCR). Real-time PCR is combined with reverse transcription polymerase chain reaction (RT) to quantify mRNA. Its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA. PCR methods are well known in the art, e.g. Higuchi, R. et al. Biotechnology, 10:413-417, 1992; Higuchi, R. et al. Biotechnology, 11:1026-30, 1993. Quantitation of mRNA by northern blotting, ribonuclease protection assay (RPA) and in situ hybridization may also be used. Standard methods and devices of detection are employed in these methods, including reading the samples containing amplified or labeled nucleic acids in devices that detect fluorescence, light, radioactive decay, or any other detectable moiety used in the methods. Such methods and devices are well known in the art.

Alternatively, protein can be isolated from the sample and the amount of TDO2 protein can be detected and quantified by any means known in the art, for example by using an antibody specific for the TDO2 protein, or by using mass spectrometry according to established methods. Examples of assays for the protein(s) are immunoassays, e.g., an ELISA assay. In addition, the sample may be analyzed for enzyme activity of the TDO2 protein, such as using the methods shown in the examples herein. These techniques are well known in the art. Standard methods and devices of detection are employed in these methods, including reading the samples containing protein or protein complexes, or enzymatic substrates or products, including detectably labeled substrates or products in devices that detect fluorescence, light, radioactive decay, or any other detectable moiety used in the methods. Such methods and devices are well known in the art.

As shown herein, control samples may contain trace amounts of TDO2 RNA or protein, and therefore the methods can include the use of a cutoff value to aid in the diagnosis or classification of the subject as having cancer or as being at risk for having cancer. The skilled person can determine such a cutoff value using control cells or sample(s), which may be control cells or sample(s) that have been determined previously, as will be known in the art. Thus in certain embodiments, the subject is diagnosed as having cancer or as being at risk for having cancer if the amount of TDO2 in the sample is determined to be more than 0.3 TDO2 molecules per cell, more than 0.4 TDO2 molecules per cell, more than 0.5 TDO2 molecules per cell, more than 0.6 TDO2 molecules per cell, more than 0.7 TDO2 molecules per cell, more than 0.8 TDO2 molecules per cell, more than 0.9 TDO2 molecules per cell, more than 1.0 TDO2 molecules per cell, more than 1.1 TDO2 molecules per cell, more than 1.2 TDO2 molecules per cell, more than 1.3 TDO2 molecules per cell, more than 1.4 TDO2 molecules per cell, more than 1.5 TDO2 molecules per cell, more than 1.6 TDO2 molecules per cell, more than 1.7 TDO2 molecules per cell, more than 1.8 TDO2 molecules per cell, more than 1.9 TDO2 molecules per cell, more than 2.0 TDO2 molecules per cell, more than 2.1 TDO2 molecules per cell, more than 2.2 TDO2 molecules per cell, more than 2.3 TDO2 molecules per cell, more than 2.4 TDO2 molecules per cell, more than 2.5 TDO2 molecules per cell, more than 2.6 TDO2 molecules per cell, more than 2.7 TDO2 molecules per cell, more than 2.8 TDO2 molecules per cell, more than 2.9 TDO2 molecules per cell, more than 3.0 TDO2 molecules per cell, more than 3.1 TDO2 molecules per cell, more than 3.2 TDO2 molecules per cell, more than 3.3 TDO2 molecules per cell, more than 3.4 TDO2 molecules per cell, more than 3.5 TDO2 molecules per cell, more than 3.6 TDO2 molecules per cell, more than 3.7 TDO2 molecules per cell, more than 3.8 TDO2 molecules per cell, more than 3.9 TDO2 molecules per cell, more than 4 TDO2 molecules per cell, more than 5 TDO2 molecules per cell, more than 6 TDO2 molecules per cell, more than 7 TDO2 molecules per cell, more than 8 TDO2 molecules per cell, more than 9 TDO2 molecules per cell, more than 10 TDO2 molecules per cell, more than 11 TDO2 molecules per cell, more than 12 TDO2 molecules per cell, more than 13 TDO2 molecules per cell, more than 14 TDO2 molecules per cell, more than 15 TDO2 molecules per cell, more than 16 TDO2 molecules per cell, more than 17 TDO2 molecules per cell, more than 18 TDO2 molecules per cell, more than 19 TDO2 molecules per cell, more than 20 TDO2 molecules per cell, or more than 30, 40, 50, 60, 70, 80, 90, or 100 TDO2 molecules per cell.

Also provided are kits for assaying the presence and/or level of TDO2 RNA or proteins. The kits include reagents that are used to contact samples in the diagnostic methods described herein, such as TDO2-specific amplification primers, TDO2-specific hybridization probes, enzymes, nucleotide mixes, anti-TDO2 antibodies or antigen-binding fragments of such antibodies, detectable labels, and other reagents known in the art to be useful and used for the diagnostic methods described herein. The kits may also include vessels for carrying out the diagnostic assays described herein, such as tubes or microwell plates.

Optionally, kits according to some embodiments of the invention may include one or more control samples. As used herein the term “control sample” typically means a sample tested in parallel with the experimental materials, although a control sample may be tested separately from experimental materials, and may be a historical control value. Examples of control samples include, but are not limited to, samples from control body fluids or tissues and samples generated through manufacture to be tested in parallel with the experimental samples.

In some embodiments, a kit may include a positive control sample and/or a negative control sample. Typically the negative control will be based on apparently healthy individuals in an appropriate age bracket. Specific examples of positive control samples include the samples shown herein to be positive for TDO2 expression, such as liver tissue or cells. Alternatively, a positive control can comprise isolated TDO2. Specific examples of negative control samples include the samples shown herein to be negative for TDO2 expression, such as skin, breast, blood or bladder tissue or cells.

The foregoing kits can include instructions or other printed material on how to use the various components of the kits for diagnostic purposes, including use of one or more cutoff values or scores.

Small Molecules

Preferred tryptophan enhancing agents are small molecule TDO2 inhibitors, including 680C91 ((E)-6-fluoro-3-[2-(3-pyridyl)vinyl]-1H-indole), 709W92 ((E)-6-fluoro-3-[2-(4-pyridyl)vinyl]-1H-indole), (M. Salter, et al., 1995. Biochem. Pharmacol. 49:1435-1442; M. Salter, et al., 1994. Neuropharmacol. 34:217-227), 540C91 ((E)-3-[2-(4′-pyridyl)-vinyl]-1H-indole) (J F Reinhard, et al., 1996. Biochem. Pharmacol. 51:159-163), Tolmetin, 2-[1-methyl-5-(4-methylbenzoyl)-pyrrol-2-yl]acetic acid, Sulindac 2-[6-fluoro-2-methyl-3-[(4-methylsulfinylphenyl)methylidene]inden-1-yl]-acetic acid (Dairam et al., 2006. Life Sci. 79:2269-74); and small molecule IDO inhibitors, including 1-methyl-DL-tryptophan (1MT) (Cady and Sono, 1991, Arch. Biochem. Biophys.291:326-333), 1-methyl-L-tryptophan, β-(3-benzofuranyl)-DL-alanine and β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, 5-bromoindoxyl diacetate, annulins A, B, and C (Pereira A et al., 2006, J. Nat. Prod. 69:1496-99), Brassinin derivatives (Gaspari P., 2006, J. MedChem. 49:684-92), necrostatin 1/methylthiohydantoine-tryptophan(5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan) (Muller A J, 2005, Nat. Med. 11:312-9), naphtoquinones (Kumar S., et al., 2008, J. MedChem. 51:1706-18), p-Coumarinic acid ((E)-3-(4-Hydroxyphenyl)-2-propenoic acid) (Kim et al., 2007, Int. Immunopharm. 7:805-15), Rosmarinic acid((2R)-2-[[(2E)-3-(3,4-Dihydroxyphenyl)-1-oxo-2-propenyl]]oxy)-3-(3,4-dihydroxyphenyl)propanoic acid) (Lee H. et al., 2006, Biochem. Pharm. 73:1412-21), Epigallocatechin (Jeong Y. et al., 2007, Biochem. Biophys. Res. Comm. 354:1004-9). Such inhibitors are commercially available. Additional IDO inhibitors are described for example, in US Patent publication No. US 2007-0173524, such as 5-bromo-4-chloroindoxyl 1,3-diacetate. The tryptophan enhancing agent preferably is not tryptophan.

As shown in the Examples, many cancers unexpectedly have been found to express tryptophan 2,3-dioxygenase (TDO2). It has previously been shown that many cancers also express indoleamine 2,3-dioxygenase (IDO) (Uyttenhove, C., L. Pilotte, I. Theate, V. Stroobant, D. Colau, N. Parmentier, T. Boon, and B. J. Van den Eynde. 2003. Nat. Med. 9:1269-1274). Some cancers may also express both enzymes.

In certain embodiments, TDO2 inhibitors are specific for TDO2 activity and do not affect IDO enzymatic activity, such as for example 540C91 ((E)-3-[2-(4′-pyridyl)-vinyl]-1H-indole), which has very low affinity to IDO, i.e. IC₅₀=1.15 mM (J F Reinhard, et al., 1996. Biochem. Pharmacol. 51:159-163). As mentioned above, TDO2 has no sequence similarity with IDO, and structural and biochemical differences of TDO2 relative to IDO and other monooxygenases enhance the specificity of inhibitors and increase the likelihood of identifying additional TDO2- and IDO-specific small molecule inhibitors (C G Prendergast, 2007, Cancer Immune Therapy, 347-368).

TDO2 inhibitors of the invention also include agents that change the concentration or distribution of cellular heme in ways that make heme less available to the TDO2 enzyme. TDO2 contains two heme molecules which are essential for its catalytic activity. In the presence of tryptophan an apoezyme-tryptophan complex forms that has a higher affinity for heme than the apoenzyme alone, and the complex competes more successfully with other intracellular heme-binding proteins. In one embodiment of the invention TDO2 inhibitors compete effectively with TDO2 for heme and therefore limit the availability of heme for TDO2. In other embodiments TDO2 inhibitors limit the availability of heme by degrading or catabolizing heme. For example, heme oxygenase I (HSP32) oxidatively cleaves heme molecules. Heme oxygenase I can be induced by reducing glutathione (GSH) through diethylmaleate (DEM) treatment or through L-buthionine-(S,R)-sulfoximine (BSO), a specific inhibitor of GSH synthesis (Ewing J F, J Neurochem. 1993, 60:1512-19). Induction of heme oxygenase I may limit heme availability to TDO2 and may inhibit TDO2 catalytic activity, which is dependent on heme molecules.

Antibodies

In certain embodiments TDO2 inhibitors are antibodies, or antigen-binding fragments thereof that specifically bind to the TDO2 polypeptide, which leads to a reduction in the catalytic activity of the TDO2 polypeptide, and in turn to a reduction in degradation of tryptophan and thus to an elevation of local tryptophan levels.

The antibodies of the present invention are prepared by any of a variety of methods, including administering a protein, fragments of a protein, cells expressing the protein or fragments thereof and the like to an animal to induce polyclonal antibodies. The present invention also provides methods of producing monoclonal antibodies to TDO2. The production of monoclonal antibodies is performed according to techniques well known in the art. It is well-known in the art that only a small portion of an antibody molecule, the paratope, is involved in the binding of the antibody to its epitope (see, in general, Clark, W. R., 1986, The Experimental Foundations of Modern Immunology, Wiley & Sons, Inc., New York; Roitt, I., 1991, Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205. Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans. Thus, as will be apparent to one of ordinary skill in the art, the present invention also provides for F(ab′)2, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab')2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies, domain antibodies and heavy chain antibodies.

Thus, the invention involves polypeptides of numerous size and type that bind specifically to TDO2 and inhibit its functional activity. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

Several TDO2-specific polyclonal and monoclonal antibodies are commercially available, e.g. from Abnova or Novus Biologicals.

Short Interfering Nucleic Acids

In certain embodiments TDO2 inhibitors are siRNAs specific for a TDO2 gene transcript, wherein the siRNAs reduce the amount of TDO2 mRNA and TDO2 protein in the TDO2 expressing cell, preferably a cancer cell.

Inhibitor molecules that are short interfering nucleic acids (siNA), which include, short interfering RNA (siRNA), double-stranded RNA (dsRNA), micro-RNA (miRNA), and short hairpin RNA (shRNA) molecules are used to inhibit the expression of target genes. The siNAs of the present invention, for example siRNAs, typically regulate gene expression via target RNA transcript cleavage/degradation or translational repression of the target messenger RNA (mRNA). In one embodiment siRNAs are exogenously delivered to a cell. In a specific embodiment siRNA molecules are generated that specifically target TDO2.

A short interfering nucleic acid (siNA) of the invention can be unmodified or chemically-modified. A siNA of the instant invention can be chemically synthesized, expressed from a vector or enzymatically synthesized. The instant invention also features various chemically-modified synthetic short interfering nucleic acid (siNA) molecules capable of inhibiting gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. For example, in some cases, siRNAs are modified to alter potency, target affinity, the safety profile and/or the stability to render them resistant or partially resistant to intracellular degradation. Modifications, such as phosphorothioates, for example, can be made to siRNAs to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006). siRNAs with amide-linked oligoribonucleosides have been generated that are more resistant to S1 nuclease degradation (Iwase Ret al. 2006 Nucleic Acids Symp Ser 50: 175-176). In addition, modification of siRNA at the 2′-sugar position and phosphodiester linkage confers improved serum stability without loss of efficacy (Choung et al., Biochem. Biophys. Res. Commun. 342(3):919-26, 2006). In one study, 2′-deoxy-2′-fluoro-beta-D-arabinonucleic acid (FANA)-containing antisense oligonucleotides compared favorably to phosphorothioate oligonucleotides, 2′-O-methyl-RNA/DNA chimeric oligonucleotides and siRNAs in terms of suppression potency and resistance to degradation (Ferrari N et a. 2006 Ann NY Acad Sci 1082: 91-102).

In some embodiments an siNA is an shRNA molecule encoded by and expressed from a genomically integrated transgene or a plasmid-based expression vector. Thus, in some embodiments a molecule capable of inhibiting gene expression is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems. Examples of making and using such hairpin RNAs for gene silencing in mammalian cells are described in, for example, (Paddison et al., Genes Dev, 2002, 16:948-58; McCaffrey et al., Nature, 2002, 418:38-9; McManus et al., RNA 2002, 8:842-50; Yu et al., Proc Natl Acad Sci USA, 2002, 99:6047-52).

One embodiment herein contemplates the use of gene therapy to deliver one or more expression vectors, for example viral-based gene therapy, encoding one or more small interfering nucleic acids, capable of inhibiting expression of TDO2. As used herein, gene therapy is a therapy focused on treating genetic diseases, such as cancer, by the delivery of one or more expression vectors encoding therapeutic gene products, including shRNAs, to diseased cells. Methods for construction and delivery of expression vectors will be known to one of ordinary skill in the art.

TDO2-specific shRNAs are commercially available, for example from Sigma/Aldrich or OriGene. The present invention, thus, contemplates in vitro use of TDO2 siRNAs (shRNAs, etc.) as well as in vivo pharmaceutical preparations containing siRNAs (shRNAs, etc.) that may be modified siRNAs (shRNAs, etc.) to increase their stability and/or cellular uptake under physiological conditions, that specifically target nucleic acids encoding TDO2 enzyme, together with pharmaceutically acceptable carriers.

Antisense Nucleic Acids

In certain embodiments TDO2 inhibitors are antisense nucleic acids. Antisense nucleic acids include short oligonucleotides as well as longer nucleic acids. Preferably the antisense nucleic acids are complementary to and bind to portions of the TDO2 coding sequence or 5′ nontranslated sequence, thereby inhibiting translation of functional TDO2 polypeptide. Other antisense nucleic acids which reduce or block TDO2 transcription are also useful.

Thus the invention embraces antisense oligonucleotides that selectively bind to a nucleic acid molecule encoding TDO2, to reduce the expression (transcription or translation) of TDO2. As used herein, the term “antisense oligonucleotide” describes an oligonucleotide that is an oligoribonucleotide, oligodeoxyribonucleotide, modified oligoribonucleotide, or modified oligodeoxyribonucleotide which hybridizes under physiological conditions to DNA comprising the TDO2 gene, e.g., human TDO2 mRNA transcript (NM_{—}005651, BC005355, U32989) and, thereby, inhibits the transcription of that gene and/or the translation of that mRNA.

Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon the sequences of TDO2 nucleic acids, including allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases.

Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. Finally, one of ordinary skill in the art may easily derive cDNA sequences and genomic DNA corresponding to TDO2 from databases and published literature. Thus, the present invention also provides for antisense oligonucleotides which are complementary to the genomic DNA corresponding to nucleic acids encoding TDO2. Similarly, antisense to allelic or homologous cDNAs and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates in vitro use of TDO2 antisense molecules as well as in vivo pharmaceutical preparations containing modified antisense molecules that are complementary to and hybridizable with, under physiological conditions, nucleic acids encoding TDO2 enzyme, together with pharmaceutically acceptable carriers.

In another embodiment, the antisense nucleic acids of the invention may be produced by expression in cells by expression vectors introduced therein. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. According to this embodiment, cells are genetically engineered by the introduction into the cells of heterologous DNA (RNA) encoding antisense TDO2 nucleic acid. The antisense TDO2 nucleic acid is placed under operable control of transcriptional elements to permit the expression of the antisense TDO2 nucleic acid in the host cell.

Preferred systems for mRNA expression in mammalian cells are those such as pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr Virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Additional vectors for delivery of antisense TDO2 nucleic acid will be known to one of ordinary skill in the art.

Various techniques may be employed for introducing antisense TDO2 nucleic acids into cells in accordance with the invention, depending on whether the nucleic acids are introduced in vitro or in vivo in a host. Such techniques include transfection of nucleic acid-CaPO₄ precipitates, transfection of nucleic acids associated with DEAE, transfection or infection with viruses including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, adenovirus or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

Expansion of T Cells

In certain embodiments TDO2 or TDO2 and IDO inhibitors are used to expand T cells in vitro, wherein TDO2 or TDO2 and IDO inhibitors described herein and/or tryptophan are added to the culture. Expansion of T cells can be carried out in a variety of different culture vessels and under different culture conditions. Other standard T cell culture protocols which differ in components or conditions (e.g., serum-free culture, addition of different cytokines, growth factors or nutrients, etc.) also can be modified in accordance with the invention.

Other types of cells in addition to T cells can benefit from being grown in the presence of tryptophan enhancing agents, e.g. in growth medium. Most growth media contain fixed amounts of tryptophan as an essential amino acid for cell metabolism. As described herein, T cells have an unexpected requirement for additional tryptophan for increased proliferation, which can be provided in growth medium by adding tryptophan to the medium or, as shown herein, by inhibiting enzymes that catabolize tryptophan. The proliferation of many kinds of cells that cannot synthesize tryptophan will benefit from the inclusion of tryptophan enhancing agents in growth media in addition to the standard nutrients which are well known in the art.

Increasing the proliferation of cells in vitro, as used herein, means increasing the number of cells by at least about 10% relative to the number of cells that are present in a parallel control population of cells that are subjected to the same conditions as the TDO2 or TDO2 and IDO inhibitor-treated population with the exception that such control population is not contacted with the either TDO2 or TDO2 and IDO inhibitors. Preferably, the number of cells are increased at least about 50%, most preferably the number of cells are increased at least about 100%.

The use of TDO2 or TDO2 and IDO inhibitors to enhance T cell culture also can increase the effector properties of the T cells, e.g., cytolytic activity, at least for some T cell clones cultured in the presence of one or more tryptophan enhancing agents. The invention includes methods for increasing activity of T cells by culturing the T cells in the presence of an effective amount of tryptophan enhancing agents to increase the activity of the T cells. In these methods, T cell activity is increased at least about 10%, preferably at least about 25%, more preferably at least about 50%, and most preferably at least about 100%.

The time period in which the number of T cells are increased can be adjusted according to the needs of the person culturing the T cells, and can be, at least in part, a function of the cell type (e.g., T cell clone) and the specific culture conditions used (growth medium, serum, cytokines, culture vessel, etc.) and/or the desired outcome (e.g., increased proliferation, increased activity, etc.). In general, this time period ranges from about 7 days (for short term expansion of T cells) to several weeks.

Routine procedures known to those of ordinary skill in the art can be used to determine the number of cells in culture as a function of increasing incubation time of the cultured cells with the TDO2 or TDO2 and IDO inhibitors (and/or tryptophan). Typically, expansion of the T cells in culture (increase in cell number) is measured by counting the cell numbers according to standard methods, for example, determining the actual cell numbers using a hematocytometer or cell counter or measuring incorporation of a specific dye. T cells also can be labeled using specific labeled antibodies and counted using an automated devices such as a fluorescence activated cell sorter (FACS). Thus, the optimization of the particular growth conditions and selection of the amounts of TDO2 or TDO2 and IDO inhibitors (i.e. one or more TDO2 or TDO2 and IDO inhibitors and/or tryptophan) that are necessary to achieve the above-noted fold increases in cell numbers (or increase in T cell activity) are determined using no more than routine experimentation. Such routine experimentation involves, for example, (i) varying the amount of a tryptophan enhancing agent at constant incubation time; (ii) varying the incubation time at constant amounts of tryptophan enhancing agent; (iii) applying the foregoing optimization experiments to determine the particular conditions necessary to achieve a pre-selected fold increase in T cell number; and (iv) varying other factors including, for example, the identity or the state of the tryptophan enhancing agent (e.g., soluble or immobilized), to optimize the culture conditions to achieve the desired results. Similar routine experimental studies can be carried out using animal models for optimization of in vivo use of tryptophan enhancing agents.

T cells that are grown in accordance with the invention can be used in a variety of in vitro and in vivo applications. For example, generating larger numbers of T cells will find application in the field of drug testing and for in vitro study of T cell biology. T cells cultured according to the invention also can be used for therapeutic purposes in vivo. For example, T cells isolated from a subject can be cultured together with TDO2 or TDO2 and IDO inhibitors in vitro (i.e. ex vivo) for expansion and eventual return to the subject. “Ex vivo” refers to in vitro culturing of cells that have first been removed from a subject prior to in vitro culturing and that may be expanded, and/or grown together with other cells, and/or treated with certain agents, and/or transfected with certain agents or nucleic acids, and/or additionally modified by any means in vitro and that may subsequently be returned to the subject that the original cells were removed from. Such ex vivo protocols are known in the art. Some therapeutic approaches using T cells are premised on a response by a subject's immune system, leading to lysis of antigen presenting cells, such as cancer cells which present one or more cancer associated antigens. One such approach is the administration of autologous cytolytic T lymphocytes (CTLs) specific to a complex of a cancer associated antigen and a MHC molecule (major histocompatibility complex; also referred to as HLA, human leukocyte antigen) to a subject with abnormal cells of the phenotype at issue. It is within the ability of one of ordinary skill in the art to develop such CTLs in vitro for use in therapeutic methods such as adoptive transfer (Greenberg, J. Immunol. 136(5): 1917, 1986; Riddel et al., Science 257: 238, 1992; Lynch et al, Eur. J. Immunol. 21: 1403-1410,1991; Kast et al., Cell 59: 603-614, 1989).

Specific production of CTL clones is well known in the art. An example of a method for T cell differentiation is presented in International Application number PCT/US96/05607. Generally, a sample of cells taken from a subject, such as blood cells, are contacted with a cell presenting the complex and capable of provoking CTLs to proliferate (e.g., cancer cells, dendritic cells). The target cell can be a transfectant, such as a COS cell transfected with nucleic acids encoding an antigen and a HLA molecule capable of presenting the antigen. These transfectants present the desired HLA/antigen complex on their surface and, when combined with a CTL of interest, stimulate its proliferation. COS cells are widely available, as are other suitable host cells. The clonally expanded autologous CTLs then are administered to the subject. Other methods for selecting antigen-specific CTL clones include the use of fluorogenic tetramers of MHC class I molecule/peptide complexes which are used to detect specific CTL clones (Altman et al., Science 274:94-96, 1996; Dunbar et al., Curr. Biol. 8:413-416, 1998). T cell clones that are specific for antigens expressed on cells infected by a pathogen also can be prepared and administered as described above.

Adoptive transfer or other methods premised on in vitro expansion of T cells are not the only forms of therapy that is available in accordance with the invention. Cytotoxic T cells (CTLs) can also be provoked in vivo, using a number of approaches. One approach is the use of non-proliferative cells expressing the complex.

Thus T cells expanded according to the invention can be used to “immunize” subjects or as “vaccines”. As used herein, “immunization” or “vaccination” means increasing or activating an immune response against an antigen. It does not require eliminationor eradication of a condition but rather contemplates the clinically favorable enhancement of an immune response toward an antigen. Generally accepted animal models can be used for testing of immunization against cancer using a T cells expanded according to the invention. For example, human cancer cells can be introduced into a mouse to create a tumor, and T cells that were expanded in cultures including tryptophan enhancing agents by the methods described herein can be administered to the mouse. The effect on the cancer cells (e.g., reduction of tumor size) can be assessed as a measure of the effectiveness of the cancer associated antigen nucleic acid immunization. Methods for increasing an immune response with T cells, including formulation of a T cell composition and selection of doses, route of administration and the schedule of administration are well known in the art. The tests also can be performed in humans, where the end point can be to test for the presence of enhanced levels of circulating cytotoxic T cells (CTLs) against cells bearing the antigen.

Thus it will be appreciated that one or more tryptophan enhancing agents can be administered as a component of an immune response modulation composition. As used herein, an immune response modulation composition is a composition administered to a subject to increase an immune response mediated by T cells. The immune response modulation composition can include T cells, antigens (e.g., peptides, proteins), nucleic acids encoding antigens, etc. which stimulate an immune response. The T cell mediated immune response induced or increased by any of these immune response modulation composition will be favorably modulated by inclusion of one or more tryptophan enhancing agents as part of the immune response modulation composition.

Compositions

The compositions of the invention are administered to a subject in effective amounts. An “effective amount” is that amount of a tryptophan enhancing agent composition that alone, or together with further doses, produces the desired response, e.g. increases proliferation and/or activity of T cells, and/or a desired improvement in the condition or symptoms of the condition, e.g., for cancer this is a reduction in cellular proliferation or metastasis. This can be monitored by routine methods known to one of ordinary skill in the art. The amount effective can be the amount of a single agent that produces a desired result or can be the amount of two or more agents in combination. Such amounts can be determined with no more than routine experimentation.

It is believed that doses ranging from 1 nanogram/kilogram to 100 milligrams/kilogram, depending upon the mode of administration, will be effective. The absolute amounts administered in vivo may depend, of course, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons. In the event that a response in a subject is insufficient at the initial doses applied, higher doses (or effectively higher doses by a different, more localized delivery route) may be employed to the extent that patient tolerance permits.

When administered in vivo, the compositions of the present invention can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The compositions used in the foregoing methods preferably are sterile and contain an effective amount of one or more tryptophan enhancing agents for producing the desired response in a unit of weight or volume suitable for addition to a cell culture or administration to a subject. As used herein, a subject is a human or non-human animal, including non-human primates, mice, rats, cows, pigs, horses, sheep, goats, dogs, cats, etc. Preferably the subject is a human.

A “subject (person or patient) having a cancer” is a subject, person or patient that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. Cancers also include cancer of the blood and larynx.

A “subject (person or patient) suspected of having a cancer” as used herein is a subject, person or patient who may show some clinical or other indications that may suggest to an observer that the subject, person or patient may have cancer. The subject, person or patient suspected of having cancer need not have undergone any tests or examinations to confirm the suspicion. It may later be established that the subject, person or patient suspected of having cancer indeed has cancer.

A “subject (person or patient) at risk of developing a cancer” as used herein is a subject, person or patient who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is treated with a TDO2 or TDO2 and IDO inhibitor of the invention the subject's immune system may be able to kill the cancer cells as they develop.

When administered to a subject, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, naphthalene-2-sulphonic, and benzene sulphonic, and the like. Also, pharmaceutically-acceptable salts can'be prepared as alkaline metal or alkaline earth salts, such as sodium, potassium or calcium salts.

The pharmaceutical compositions may contain suitable buffering agents, including: acetic acid in a salt; citric acid in a salt; boric acid in a salt; and phosphoric acid in a salt.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V). Suitable preservatives include benzalkonium chloride (0.003-0.03% W/V); chlorobutanol (0.3-0.9% W/V); parabens (0.01-0.25% W/V) and thimerosal (0.004-0.02% W/V).

A tryptophan enhancing agent, e.g. a TDO2 or TDO2 and IDO inhibitor composition, may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being comingled with the molecules of the present invention, and with each other, in a manner such that there is no interaction which would substantially impair the desired pharmaceutical efficacy.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product. Among the acceptable vehicles and solvents that may be employed are water, Ringer's solution, and isotonic sodium chloride solution, but are not so limited. In addition, sterile, fixed oils are conventionally employed as a solvent or suspending medium. For this purpose any bland fixed oil may be employed including synthetic mono-or di-glycerides. Carrier formulation suitable for oral, subcutaneous, intravenous, intramuscular, etc. administrations can be found in Remington's Pharmaceutical Sciences, Mack Publishing Co., Easton, Pa.

The therapeutics provided herein, for example tryptophan enhancing agents, can be administered in vivo by any conventional route, including injection or by gradual infusion over time. The administration may, for example, be oral, intravenous, intraperitoneal, intramuscular, intracavity, subcutaneous, intrasternal, transdermal and intratumoral. Other modes of administration include mucosal, rectal, vaginal, sublingual, intranasal, intratracheal, inhalation, ocular, and transdermal.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions may conveniently be presented in unit dosage form and may be prepared by any of the methods well known in the art of pharmacy.

Other delivery systems can include time-release, delayed release or sustained release delivery systems. Such systems can avoid repeated administrations of the compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di- and triglycerides; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. Nos. 4,748,034 and 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni). In addition, a pump-based hardware delivery system can be used, some of which are adapted for implantation.

Controlled release of TDO2 or TDO2 and IDO inhibitors can also be achieved with appropriate excipient materials that are biocompatible and biodegradable. These polymeric materials which effect slow release of the TDO2 or TDO2 and IDO inhibitors of the invention may be any suitable polymeric material for generating particles, including, but not limited to, nonbioerodable/non-biodegradable and bioerodable/biodegradable polymers.

Such polymers have been described in great detail in the prior art. They include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate.

Examples of preferred non-biodegradable polymers include ethylene vinyl acetate, poly(meth) acrylic acid, polyamides, copolymers and mixtures thereof. Examples of preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. The most preferred polymers are polyesters, polyanhydrides, polystyrenes and blends thereof.

### Anti-Cancer Agents and Immune Modulators

In certain embodiments TDO2 or TDO2 and IDO inhibitors of the invention can be administered as singly active agents or can be combined with anti-cancer agents and/or immune modulators. These additional agents may enhance the therapeutic and prophylactic effects of the TDO2 or TDO2 and IDO inhibitors. IDO has successfully been used in combination with anti-cancer agents in tumor models (see, WO 2004/094409 or US 2007-0173524, and A J Muller, 2005, Cancer Res. 65:8065-68).

“Anti-cancer agents” can include cytotoxic agents and agents that act on tumor neovasculature. Cytotoxic agents include cytotoxic radionuclides, chemical toxins and protein toxins. The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as 186Rh, ¹⁸⁸Rh, ¹⁷⁷Lu, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes 125I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cisplatin, etoposide, bleomycin and 5-fluorouracil. Toxins also include poisonous lectins, plant toxins such as ricin, abrin, modeccin, botulina and diphtheria toxins. Of course, combinations of the various toxins are also provided thereby accommodating variable cytotoxicity. Other chemotherapeutic agents are known to those skilled in the art. Anti-cancer agents can be bound to tumor-specific antibodies or antigen-binding fragments thereof to generate cytotoxic antibodies.

Agents that act on the tumor vasculature (”anti-vasculature agents” and “anti-angiogenesis agents”) can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein), interferon inducible protein 10 (U.S. Pat. No. 5,994,292), and the like.

Additional chemotherapeutic agents are for instance, vincristine, adriamycin, non-sugar containing chloroethylnitrosoureas, dacarbazine, taxol, fragyline, Meglamine GLA, valrubicin, carmustaine and poliferposan, MMI270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/NX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCI, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), or Teniposide (VM-26), but are not so limited.

Other agents which stimulate the immune response of the subject can also be administered to the subject in an immune response modulation composition. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF and IL-18. Thus cytokines can be administered in conjunction with T cells and adjuvants to increase the immune response to the antigens.

There are a number of immune response potentiating compounds that can be used in vaccination protocols. These include co-stimulatory or antigenic molecules provided in either peptide or protein form or as nucleic acids. Such co-stimulatory molecules include the B7-1 and B7-2 (CD80 and CD86 respectively) molecules which are expressed on dendritic cells (DC) and interact with the CD28 molecule expressed on the T cell. Another co-stimulatory molecule is the ICOS protein. These interactions provide co-stimulation (signal 2) to an antigen/MHC/TCR (T cell receptor) stimulated (signal 1) T cell (“antigen-specific T lymphocyte”), increasing T cell proliferation and effector function.

Thus, TDO2 or TDO2 and IDO inhibitors of the invention singly or combined with anticancer agents described herein can additionally be combined with immune modulators such as α-interferon, tumor necrosis factor alpha (TNFα), CD40L, B7, B7RP1, anti-CD40, anti-CD38, anti-ICOS, 4-IBB ligand, dendritic cell cancer vaccine, IL2, IL12, ELC/CCL19, SLC/CCL21, MCP-1, IL-4, IL-18, IL-15, MDC, M-CSF, IL-3, GM-CSF, Il-13, anti-IL-10, bacterial lipopolysaccharides, and poly-CpG DNA.

Adjuvants also can be added to the immune response modulation compositions, comprising for example TDO2 or TDO2 and IDO inhibitors of the invention, singly or combined with anticancer agents, and/or tumor antigens, and/or other chemotherapeutic agents and/or immune modulators described herein. Many kinds of adjuvants are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham); combinations of DQS21/MPL mixed in ratios of about 1:10 to 10:1; QS-7, QS-17, QS-18, and QS-L1 (So et al., Mol. Cells 7:178-186, 1997); CpG; incomplete Freund's adjuvant; complete Freund's adjuvant; montanide; and various water-in-oil emulsions prepared from biodegradable oils such as squalene and/or tocopherol. Other adjuvants are known in the art and can be used in the invention (see, e.g. Goding, Monoclonal Antibodies: Principles and Practice, 2nd Ed., 1986). Methods for the preparation of mixtures or emulsions of antigens and adjuvant are well known to those of skill in the art of vaccination.

Certain anti-cancer agents may be linked to targeting molecule which specifically interact with a cancer cell or a tumor. For instance, the targeting molecule may be a protein or other type of molecule that recognizes and specifically interacts with a tumor antigen.

Tumor-antigens include Melan-A/MART-1, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin and γ-catenin, p120ctn, gp100^{Pmell17}, PRAME, NY-ESO-1, brain glycogen phosphorylase, SSX-1, SSX-2 (HOM-MEL-40), SSX-4, SSX-5, SCP-1, CT-7, cdc27, adenomatous polyposis coli protein (APC), fodrin, PIA, Connexin 37, Ig-idiotype, p15, gp75, GM2 and GD2 gangliosides, viral products such as human papilloma virus proteins, Smad family of tumor antigens, lmp-1, EBV-encoded nuclear antigen (EBNA)-1, and c-erbB-2. Additional tumor antigens are known in the art, for example Scanlan M. J., Simpson A. J. G., Old L. J., Cancer Immunity, 4:1, 2004; Scanlan M. J., Gure A. O., Jungbluth A. A., Old L. J., Chen Y. T., Immunol Rev, 188:22-32, 2002; Chen Y. T. et al., Proc Nat Ac Sci, 102:7940-45, 2005.

Additional immunotherapeutic agents are for instance, Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab or ImmuRAIT-CEA, but are not so limited.

The present invention is further illustrated by the following Examples, which in no way should be construed as further limiting.

### EXAMPLES

### Example 1

### Detection of Tryptophan 2,3-dioxygenase (TDO2) in Cancer Cell Lines and Tumor Tissue

We studied the expression of TDO2 in tumor tissue and cancer cell lines by RT-PCR. We chose a set of primers matching the sequence of both human and mouse TDO2, as follows:

| | |
|---|---|
| sense primer: | |
| 5′-TTGGACTTCAATGACTTCAGAGA-3′ | (SEQ ID NO: 1) |
| antisense primer: | |
| 5′-TGCCCAGCATTCTGTGC-3′ | (SEQ ID NO: 2) |

We used standard conditions for the reverse transcription, and the following conditions for the PCR amplification:

94° C. for 3 min

35 cycles (94° C. for 1 min/56° C. for 1 min/72° C. for 1 min)

72° C. for 10 min.

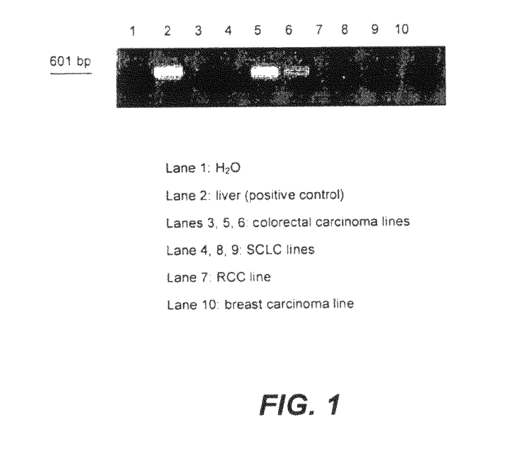

We used RNA from human liver as a positive control sample. Exemplary results are presented in FIG. 1. As indicated in Table 1, 40 tumor lines out of 63 tested scored positive for TDO2 expression, at various levels. Out of 66 tumor samples tested, 56 scored positive for TDO2.

TABLE 1
Number of TDO2-positive out of the number of total cell
lines (left column) and samples (right column) tested.

| Tumor lines | | Tumor samples | |
|---|---|---|---|
| Melanoma | 9/13 | Melanoma | 5/10 |
| Colorectal adenocarcinoma | 7/10 | Colorectal adenocarcinoma | 10/10 |
| Renal cell carcinoma | 4/8 | Renal cell carcinoma | 8/9 |
| SCLC | 1/3 | NSCLC | 8/9 |
| NSCLC | 5/6 | Breast cancer | 10/10 |
| Breast cancer | 2/3 | Head and neck carcinoma | 9/10 |
| Pancreatic adenocarcinoma | 1/3 | Bladder cancer | 6/8 |
| Mesothelioma | 4/5 | | |
| Head and neck carcinoma | 6/11 | | |
| Bladder cancer | 1/1 | | |

### Example 2

### Generation of TDO2 Expressing Clones

To evaluate the effect of tumoral expression of TDO2 on tumor rejection, we used mouse tumor P815, which does not express TDO2 and can be readily rejected by mice previously immunized against the antigen encoded by P1A, a cancer-germline gene encoding the major rejection antigen of tumor P185 (Van den Eynde, B., B. Lethé, A. Van Pel, E. De Plaen, and T. Boon. 1991. The gene coding for a major tumor rejection antigen of tumor P815 is identical to the normal gene of syngeneic DBA/2 mice. J. Exp. Med. 173:1373-1384; Brändle, D., J. Bilsborough, T. Rülicke, C. Uyttenhove, T. Boon, and B. J. Van den Eynde. 1998. Eur. J. Immunol. 28:4010-4019).

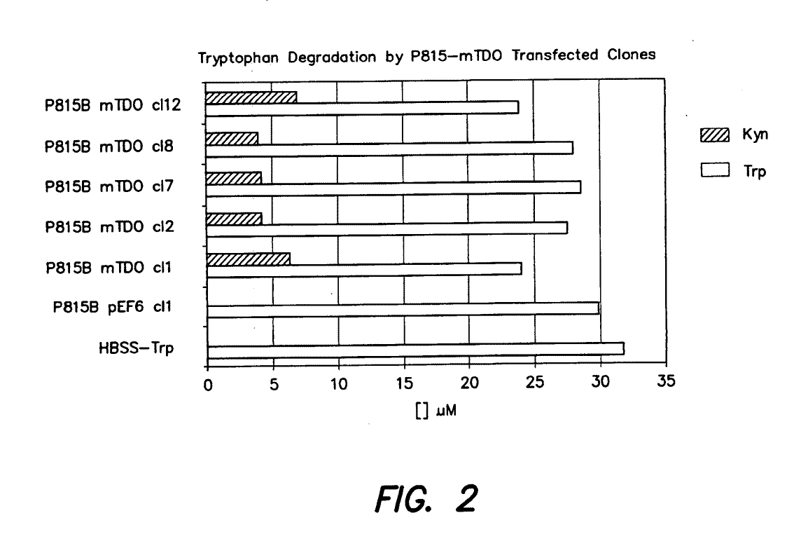

We transfected P815 cells with a plasmid construct encoding mouse TDO2 and we selected transfected clones expressing TDO2. We tested the TDO activity of the transfected clones by measuring their ability to degrade tryptophan and produce kynurenine (FIG. 2). A negative control clone transfected with an empty plasmid was also selected (P815B pEF6 cl1).

### Example 3

### TDO2 Expression Protects Tumor Cells from Immune Surveillance and Killing In Vivo

We then immunized DBA/2 mice by injecting one million living L1210.P1A.B7-1 cells in the peritoneal cavity. Four weeks later, mice were injected with 4×10⁵ cells of the P815 control clone (pEF) or with two transfected clones expressing TDO2 (clones 2 and 8).

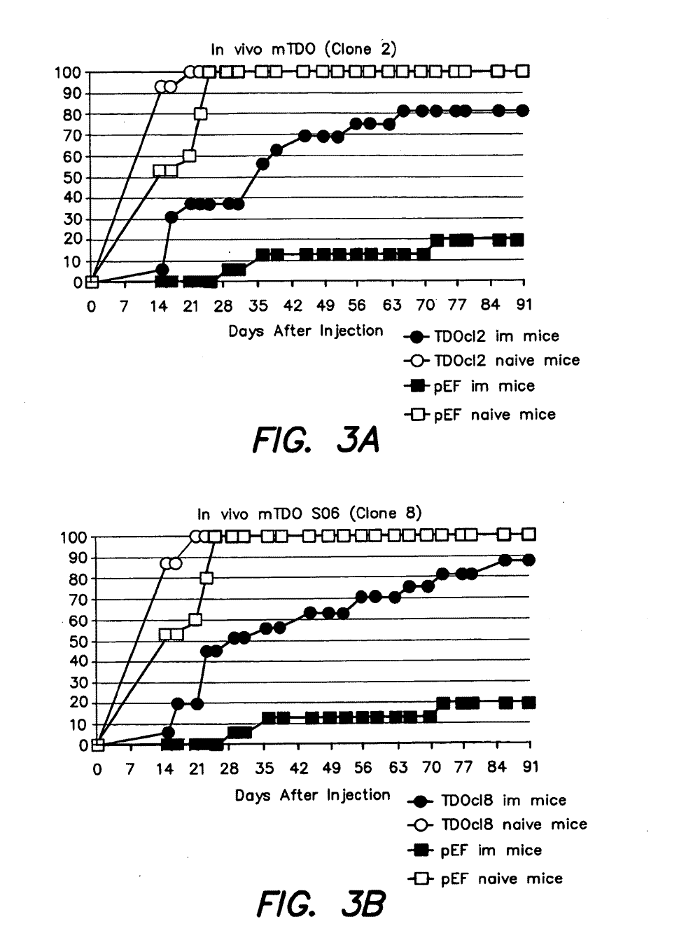

Tumor progression was monitored (FIG. 3). All clones produced progressive tumors in naive (non-immunized) control mice. Most immunized mice rejected the control P815 cells. However, TDO-expressing P815 cells produced progressive tumors in the majority of immunized mice. These results indicate that TDO2 expression by tumor cells allows these tumor cells to resist immune rejection. We therefore predict that pharmacological inhibition of TDO2 will restore tumor rejection in such mice. Given the frequent expression of TDO2 in human tumors, inhibition of TDO2 in cancer patients should boost the clinical efficacy of cancer immunotherapy.

### Example 4

### The TDO2 Inhibitor 680C91 Efficiently Blocked Tryptophan Degradation and Kynurenin Pproduction by TDO-Expressing Cells

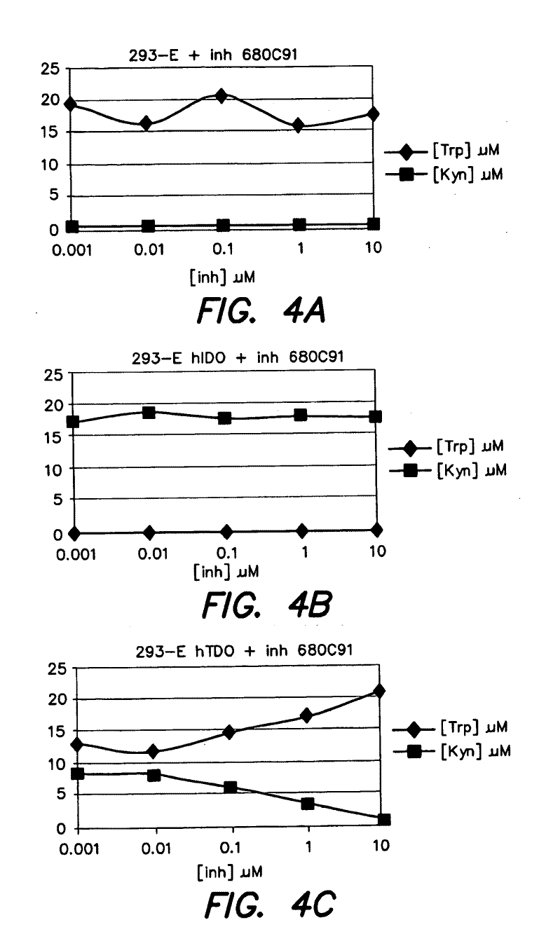

A TDO inhibitor, 680C91, was described in Salter, M., R. Hazelwood, C. I. Pogson, R. Iyer, and D. J. Madge. 1995. Biochem. Pharmacol. 49:1435-1442. We synthesized this compound and tested its capacity to block tryptophan degradation by human 293 cells transfected with TDO2. As shown on FIG. 4, 680C91 efficiently blocked tryptophan degradation and kynurenin production by TDO-expressing cells, while it failed to block tryptophan degradation and kynurenin production by IDO-expressing cells, confirming the specific inhibition of TDO2 by 680C91.

### Example 5

### Selection of Highly Expressing TDO2 Transfectants

The TDO2 inhibitor 680C91 is used to select P815 transfectants expressing higher levels of TDO2. Tryptophan depletion that counterselects highly expressing transfectants is prevented by adding the inhibitor to the culture medium.

### Example 6

### Expression of TDO in Human Tumors

We have refined our analysis of TDO expression by performing a real-time RT-PCR (Taqman).

To evaluate the level of expression of TDO2 by quantitative RT-PCR, 1/40 of the cDNA produced from 2 μg of total RNA was amplified with primers: CATGGCTGGAAAGAACTC (forward; SEQ ID NO:3) and CTGAAGTGCTCTGTATGAC (reverse; SEQ ID NO:4) in the presence of the double-dye probe: 5′-6-FAM-TTTAGAGCCACATGGATTTAACTTCTGGG-Tamra-3′ (SEQ ID NO:5). The different RNA samples were normalized by quantification of the level of expression of ACTINB using primers: GGCATCGTGATGGACTCCG (forward; SEQ ID NO:6) and GCTGGAAGGTGGACAGCGA (reverse; SEQ ID NO:7) in the presence of probe 5′-6-FAM-TCAAGATCATTGCTCCTCCTGAGCGC-TAMRA-3′ (SEQ ID NO:8). Thermal cycling and fluorescence monitoring were performed in an ABI Prism 7700 sequence detector. Thermal conditions were 10 min at 95° C. and 40 cycles of 15 sec at 95° C. and 1 min at 60° C. (TDO2) or 1 min, 30 sec at 62° C. (ACTINB). In order to estimate the number of mRNA molecules per cell, we calculated a ΔCt value for each sample and considered that each cell contains 2,000 β-actin transcripts. All values are means of duplicate determinations.

The results are summarized in Table 2.

TABLE 2

| | mRNA |
|---|---|
| | molecules |
| | TDO2/cell |
| Tumor samples | |
| Bladder carcinoma 1015 | 1.2 |
| Bladder carcinoma 1024 | 0.5 |
| Bladder carcinoma 1028 | 0.9 |
| Bladder carcinoma 1034 | 0.3 |
| Bladder carcinoma 1036 | 0.0 |
| Bladder carcinoma 1037 | 0.1 |
| Bladder carcinoma 1055 | 1.6 |
| Bladder carcinoma 1186 | 1.4 |
| Bladder carcinoma 1195 | 5.5 |
| Bladder carcinoma 1197 | 6.8 |
| Bladder carcinoma 1201 | 2.6 |
| Bladder carcinoma 1203 | 1.3 |
| Bladder carcinoma 1317 | 0.1 |
| Bladder carcinoma 1324 | 3.6 |
| Bladder carcinoma 1770 | 5.5 |
| Bladder carcinoma 1771 | 2.6 |
| Bladder carcinoma 2608 | 3.6 |
| Bladder carcinoma 2629 | 0.3 |
| Bladder carcinoma 2639 | 0.9 |
| Bladder carcinoma 2646 | 1.0 |
| Hepatocarcinoma 3445 | 4000 |
| Hepatocarcinoma 3637 | 54.4 |
| Hepatocarcinoma 3638 | 134.0 |
| Hepatocarcinoma 3789 | 1000.0 |
| Hepatocarcinoma 3862 | 116.6 |
| Hepatocarcinoma 3886 | 47.4 |
| Hepatocarcinoma 4022 | 189.5 |
| Melanoma 2384 | 16.7 |
| Melanoma 4073 | 2.2 |
| Melanoma 5661 | 2.4 |
| Melanoma 5837 | 8.4 |
| Melanoma 5838 | 0.9 |
| Melanoma 6006 | 3.0 |
| Melanoma 6161 | 2.0 |
| Melanoma 6405 | 0.9 |
| Melanoma 6620 | 5.9 |
| Melanoma 6621 | 3.4 |
| Melanoma 6647 | 4.5 |
| Melanoma 6659 | 7.3 |
| Melanoma 996 | 0.3 |
| Melanoma 6099 | 0.6 |
| Melanoma 6107 | 1.4 |
| Melanoma 6111 | 0.4 |
| Melanoma 6393 | 0.4 |
| Melanoma 6400 | 1.1 |
| Melanoma 6413 | 0.7 |
| Melanoma 6676 | 0.4 |
| Mesothelioma 2689 | 2.4 |
| Mesothelioma 3631 | 14.6 |
| Mesothelioma 483 | 0.0 |
| Mesothelioma 506 | 1.4 |
| Neuroblastoma 1251 | 1.4 |
| Neuroblastoma 2489 | 2.1 |
| Neuroblastoma 2493 | 8.4 |
| Sarcoma 1067 | 1.5 |
| Sarcoma 1063 | 1.6 |
| Sarcoma 1064 | 0.3 |
| Sarcoma 1065 | 0.0 |
| Sarcoma 2338 | 5.9 |
| Breast carcinoma 215 | 16.7 |
| Breast carcinoma 224 | 7.8 |
| Breast carcinoma 235 | 0.9 |
| Breast carcinoma 236 | 1.6 |
| Breast carcinoma 237 | 0.2 |
| Breast carcinoma 262 | 0.3 |
| Breast carcinoma 284 | 0.1 |
| Breast carcinoma 301 | 0.9 |
| Breast carcinoma 330 | 0.1 |
| Breast carcinoma 334 | 1.3 |
| Breast carcinoma 336 | 0.7 |
| Breast carcinoma 341 | 0.1 |
| Breast carcinoma 346 | 0.2 |
| Breast carcinoma 447 | 0.1 |
| Breast carcinoma 522 | 0.4 |
| Breast carcinoma 527 | 0.1 |
| Leukemia 1555 | 0.0 |
| Leukemia 1809 | 0.0 |
| Leukemia 1819 | 0.0 |
| Leukemia 1814 | 0.0 |
| Leukemia 1826 | 0.0 |
| Leukemia 1827 | 0.0 |
| Leukemia 1829 | 0.0 |
| Leukemia 1836 | 0.0 |
| Leukemia 1837 | 0.0 |
| Leukemia 1838 | 0.1 |
| Leukemia 1861 | 0.0 |
| Leukemia 1862 | 0.0 |
| Leukemia 1863 | 0.0 |
| Leukemia 1866 | 0.0 |
| Leukemia 2078 | 0.0 |
| Leukemia 2186 | 0.0 |
| Leukemia 2425 | 0.7 |
| Leukemia 2520 | 0.0 |
| Leukemia 2687 | 0.0 |
| Leukemia 2741 | 0.0 |
| Leukemia 2819 | 0.0 |
| Leukemia 3744 | 0.0 |
| Leukemia 3850 | 0.0 |
| Leukemia 4642 | 16.7 |
| Leukemia 133 | 0.0 |
| Renal cell carcinoma 2435 | 0.0 |
| Renal cell carcinoma 2519 | 0.2 |
| Renal cell carcinoma 4954 | 0.3 |
| Renal cell carcinoma 1810 | 88.4 |
| Renal cell carcinoma 550 | 0.0 |
| Renal cell carcinoma 551 | 0.0 |
| Renal cell carcinoma 552 | 0.0 |
| Colorectal carcinoma 158 | 0.0 |
| Colorectal carcinoma 210 | 0.6 |
| Colorectal carcinoma 182 | 0.0 |
| Colorectal carcinoma 427 | 0.0 |
| Colorectal carcinoma 409 | 13.6 |
| Colorectal carcinoma 455 | 0.1 |
| Colorectal carcinoma 195 | 4.8 |
| Head and Neck carcinoma 1568 | 0.1 |
| Head and Neck carcinoma 1537 | 0.2 |
| Head and Neck carcinoma 1515 | 0.0 |
| Head and Neck carcinoma 1553 | 0.0 |
| Head and Neck carcinoma 1579 | 1.8 |
| Head and Neck carcinoma 2229 | 9.0 |
| Head and Neck carcinoma 2818 | 1.4 |
| Head and Neck carcinoma 3161 | 0.0 |
| Head and Neck carcinoma 1896 | 0.3 |
| Lung carcinoma 535 | 0.3 |
| Lung carcinoma 2804 | 0.7 |
| Lung carcinoma 2602 | 0.3 |
| Lung carcinoma 132 | 1.1 |
| Lung carcinoma 1984 | 19.2 |
| Lung carcinoma 2671 | 3.6 |
| Lung carcinoma 2600 | 0.4 |
| Lung carcinoma 445 | 1.1 |
| Tumor lines | |
| Melanoma GEMO-1 | 0.0 |
| Melanoma DAGI | 3.4 |
| Melanoma MZ2-MEL | 0.2 |
| Melanoma BIJA-1 | 0.1 |
| Melanoma A375 | 0.1 |
| Melanoma DAUV-4A | 0.0 |
| Melanoma RIPA-1 | 0.6 |
| Melanoma AUMA-1 | 0.5 |
| Melanoma 2866 AUMA | 3.0 |
| Melanoma C5A1 | 0.0 |
| Melanoma 2097 CALU | 0.1 |
| Hepatoma HUH7 | 5.2 |
| Bladder carcinoma 2952 HECT | 1.5 |
| Mesothelioma 1472 HM-74 | 1.8 |
| Mesothelioma 1467 HM-69 | 3.9 |
| Breast carcinoma 5230 HS-578T | 3.9 |
| Colorectal carcinoma 173 MZ-CO2 | 17.9 |
| Colorectal carcinoma 213 SKCO11 | 50.8 |
| Colorectal carcinoma 204 FREN | 44.2 |
| Renal cell carcinoma 1825 BAYA | 22.1 |
| Renal cell carcinoma 3379 TREP | 8.4 |
| Renal cell carcinoma 4260 IDEM | 1.7 |
| Lung carcinoma 925 GLCP1 | 2.0 |
| Lung carcinoma 2599 NCI-H460 | 41.2 |
| Head and Neck carcinoma 2720 SENY | 12.7 |
| Normal tissues | |
| Liver 1765 | 707.1 |
| Liver 3168 | 406.1 |
| Liver 3208 | 1515.7 |
| Skin 2302 | 0.0 |
| Skin 5360 | 0.0 |
| Blood 4304 | 0.0 |
| Blood 5507 | 0.0 |
| Breast 2107 | 0.0 |
| Breast 2138 | 0.1 |
| Breast 5404 | 0.1 |
| Urinary Bladder 1601 | 0.3 |
| Urinary Bladder 5395 | 0.1 |

Values were calculated based on ΔCt (TDO2-β-actin), considering 2000 β-actin transcripts per cell.

### Example 7

### Enzymatic Activity of TDO2 in Human Tumors

To confirm the expression of enzymatically active TDO2, we used Head and Neck carcinoma cell line 2720 (SENY), which expresses 12.7 molecules of TDO2 mRNA/cell. We measured tryptophan consumption and kynurenine production in the supernatant of 5×10⁵ cells incubated overnight in 2 ml of HBSS containing 50 μM tryptophan. Tryptophan and kynurenine concentrations were measured by HPLC using standard methods.

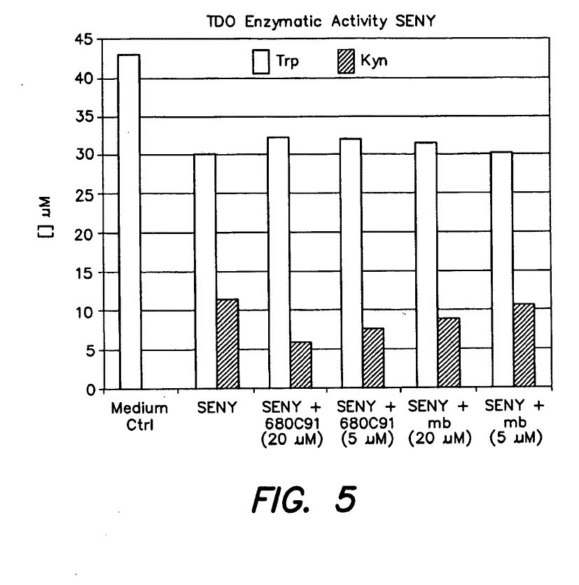

See FIG. 5 for results of this assay. To confirm that the tryptophan-degrading activity was due to TDO, TDO-specific inhibitor 680C91 was included at 5 μM or 20 μM. As control, an IDO inhibitor (mb) was also used at 5 μM or 20 μM. A tryptophan-degrading activity was detected and was clearly observed by looking at the production of kynurenine: 11 μM as compared to 0 in the control without cells. This activity was mostly attributable to TDO, as it was blocked by the TDO-specific inhibitor 680C91 (50% inhibition at 20 μM). This activity was only marginally blocked by the IDO inhibitor mb.

### Equivalents

Those skilled in the art will recognize, or be able to ascertain using no more than routine experimentation, many equivalents to the specific embodiments of the invention described herein. Such equivalents are intended to be encompassed by the following claims.

The entire contents of all of the references (including literature references, issued patents, published patent applications, and co-pending patent applications) cited throughout this application are hereby expressly incorporated by reference for the purposes or subject matter referenced herein.

We claim:

1. A method for diagnosing cancer in a subject comprising
determining the expression of tryptophan 2,3-dioxygenase (TDO2) in a subject, wherein the expression of TDO2 is determined in a subject by
obtaining a sample containing cells from the subject that is not a liver sample, and
measuring the expression of TDO2 in the sample, and
wherein the expression of TDO2 in the sample indicates that the subject has cancer.

2. The method of claim 1, wherein the determination of TDO2 expression is carried out measuring TDO2 mRNA or protein in the sample.

3. The method of claim 2, wherein the TDO2 mRNA in the sample is measured by PCR.

4. The method of claim 3, wherein the PCR is real time RT-PCR.

5. The method of claim 2, wherein the TDO2 protein in the sample is measured by an immunoassay using an antibody that specifically binds TDO2 protein.

6. The method of claim 5, wherein the assay is an ELISA assay.

7.-10. (canceled)

11. A method for treating a subject having or suspected of having a cancer, or being at risk of developing a cancer, comprising administering to the subject in need of such treatment an amount of an inhibitor effective to inhibit the activity of tryptophan 2,3-dioxygenase (TDO2).

12. The method of claim 11, wherein the cells of the cancer express tryptophan 2,3-dioxygenase (TDO2).

13. The method of claim 11, wherein the TDO2 inhibitor is selected from the group consisting of 680C91 ((E)-6-fluoro-3-[2-(3-pyridyl)vinyl]-1H-indole), 709W92 ((E)-6-fluoro-3-[2-(4-pyridyl)vinyl]-1H-indole), Sulindac (2-[6-fluoro-2-methyl-3-[(4-methylsulfinylphenyl)methylidene]inden-1-yl]-acetic acid), and 540C91 ((E)-3-[2-(4′-pyridyl)-vinyl]-1H-indole), Tolmetin (2-[1-methyl-5-(4-methylbenzoyl)-pyrrol-2-yl]acetic acid).

14. The method of claim 11, wherein the TDO2 inhibitor is a heme depleting agent, wherein the heme depleting agent reduces the availability of heme to the TDO2 polypeptide.

15. The method of claim 14, wherein the heme depleting agent is an agent that induces the expression of heme oxygenase-1 (HSP32).

16. The method of claim 15, wherein the heme oxygenase-1 inducing agent is a glutathione-reducing agent.

17. The method of claim 16, wherein the glutathione-reducing agent is selected from the group consisting of diethyl maleate (DEM) and L-buthionine-(S,R)-sulfoximine (BSO).

18.-27. (canceled)

28. The method of claim 11, further comprising administering to a subject an amount of an indoleamine 2,3-dioxygenase (IDO) inhibitor effective to inhibit IDO.

29. The method of claim 28, wherein the IDO inhibitor is selected from the group consisting of 1-methyl-DL-tryptophan, 1-methyl-L-tryptophan, β-(3-benzofuranyl)-DL-alanine, β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, 5-bromoindoxyl diacetate, 5-bromo-4-chloroindoxyl 1,3-diacetate, annulin A, annulin B, annulin C, Brassinin derivatives, necrostatin 1/methylthiohydantoine-tryptophan (5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan), naphtoquinones, p-Coumarinic acid ((E)-3-(4-Hydroxyphenyl)-2-propenoic acid), Rosmarinic acid ((2R)-2-[[(2E)-3-(3,4-Dihydroxyphenyl)-1-oxo-2-propenyl]]oxyl-3-(3,4-dihydroxyphenyl)propanoic acid), and Epigallocatechin.

30. The method of claim 11, further comprising administering one or more anti-cancer agents.

31. The method of claim 30, wherein the anti-cancer agent is selected from the group consisting of cytotoxic T cells (CTL), cytotoxic antibodies, cytotoxic or growth-inhibitory chemotherapeutic agents, and anti-vasculature or anti-angiogenesis agents.

32.-39. (canceled)

40. A method for inhibiting the growth or killing cancer cells which have evaded or have the potential to evade T cell-mediated cytolysis, comprising
contacting the cancer cells with an amount of an inhibitor of tryptophan 2,3-dioxygenase (TDO2) effective to increase T cell-mediated cytolysis of the cancer cells, wherein the cancer cells express TDO2, thereby inhibiting the growth or killing the cancer cells.

41. The method of claim 40, wherein the cancer cells express tryptophan 2,3-dioxygenase (TDO2).

42. The method of claim 40, wherein the TDO2 inhibitor is selected from the group consisting of 680C91 ((E)-6-fluoro-3-[2-(3-pyridyl)vinyl]-1H-indole), 709W92 ((E)-6-fluoro-3-[2-(4-pyridyl)vinyl]-1H-indole), Tolmetin (2-[1-methyl-5-(4-methylbenzoyl)-pyrrol-2-yl]acetic acid), and Sulindac (2-[6-fluoro-2-methyl-3-[(4-methylsulfinylphenyl)methylidene]inden-1-yl]-acetic acid), and 540C91 ((E)-3-[2-(4′-pyridyl)-vinyl]-1H-indole).

43. The method of claim 40, wherein the TDO2 inhibitor is a heme depleting agent, wherein the heme depleting agent reduces the availability of heme to the TDO2 polypeptide.

44. The method of claim 43, wherein the heme depleting agent is an agent that induces the expression of heme oxygenase-1 (HSP32).

45. The method of claim 44, wherein the heme oxygenase-1 inducing agent is a glutathione-reducing agent.

46. The method of claim 45, wherein the glutathione-reducing agent is selected from the group consisting of diethyl maleate (DEM) and L-buthionine-(S,R)-sulfoximine (BSO).

47.-56. (canceled)

57. The method of claim 40, further comprising contacting the cancer cells with an amount of an indoleamine 2,3-dioxygenase (IDO) inhibitor effective to inhibit IDO.

58. The method of claim 57, wherein the IDO inhibitor is selected from the group consisting of 1-methyl-DL-tryptophan, 1-methyl-L-tryptophan, β-(3-benzofuranyl)-DL-alanine, β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, and 5-bromoindoxyl diacetate, and 5-bromo-4-chloroindoxyl 1,3-diacetate, annulin A, annulin B, annulin C, Brassinin derivatives, necrostatin 1/methylthiohydantoine-tryptophan (5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan), naphtoquinones, p-Coumarinic acid ((E)-3-(4-Hydroxyphenyl)-2-propenoic acid), Rosmarinic acid ((2R)-2-[[(2E)-3-(3,4-Dihydroxyphenyl)-1-oxo-2-propenyl]]oxy]-3-(3,4-dihydroxyphenyl)propanoic acid), and Epigallocatechin.

59. The method of claim 40, further comprising contacting the cancer cells with one or more anti-cancer agents.

60. The method of claim 59, wherein the anti-cancer agent is selected from the group consisting of cytotoxic T cells (CTL), cytotoxic antibodies, cytotoxic or growth-inhibitory chemotherapeutic agents, and anti-vasculature or anti-angiogenesis agents.

61.-64. (canceled)

65. A pharmaceutical composition comprising
an amount of a tryptophan 2,3-dioxygenase (TDO2) inhibitor effective to inhibit TDO2 and increase local tryptophan concentrations in the presence of TDO2 polypeptide expression, and
a pharmaceutically acceptable carrier.

66. The pharmaceutical composition of claim 65, wherein the TDO2 inhibitor is selected from the group consisting of 680C91 ((E)-6-fluoro-3-[2-(3-pyridyl)vinyl]-1H-indole), 709W92 ((E)-6-fluoro-3-[2-(4-pyridyl)vinyl]-1H-indole), Tolmetin (2-[1-methyl-5-(4-methylbenzoyl)-pyrrol-2-yl]acetic acid), and Sulindac (2-[6-fluoro-2-methyl-3-[(4-methylsulfinylphenyl)methylidene]inden-1-yl]-acetic acid), and 540C91 ((E)-3-[2-(4′-pyridyl)-vinyl]-1H-indole).

67. The pharmaceutical composition of claim 65, wherein the TDO2 inhibitor is a heme depleting agent, wherein the heme depleting agent reduces the availability of heme to the TDO2 polypeptide.

68. The pharmaceutical composition of claim 67, wherein the heme depleting agent is an agent that induces the expression of heme oxygenase-1 (HSP32).

69. The pharmaceutical composition of claim 68, wherein the heme oxygenase-1 inducing agent is a glutathione-reducing agent.

70. The pharmaceutical composition of claim 69, wherein the glutathione-reducing agent is selected from the group consisting of diethyl maleate (DEM) and L-buthionine-(S,R)-sulfoximine (BSO).

71.-80. (canceled)

81. The pharmaceutical composition of claim 65, further comprising an indoleamine 2,3-dioxygenase (IDO) inhibitor effective to inhibit IDO and to increase local tryptophan concentrations in the presence of TDO2 polypeptide expression.

82. The pharmaceutical composition of claim 81, wherein the IDO inhibitor is selected from the group consisting of 1-methyl-DL-tryptophan, β-(3-benzofuranyl)-DL-alanine, β-[3-benzo(b)thienyl]-DL-alanine, 6-nitro-L-tryptophan, indole 3-carbinol, 3,3′-diindolylmethane, epigallocatecin gallate, 5-Br-4-CL-indoxyl 1,3-diacetate, 9-vinylcarbazole, acemetacin, 5-bromo-DL-tryptophan, and 5-bromoindoxyl diacetate, and 5-bromo-4-chloroindoxyl 1,3-diacetate annulin A, annulin B, annulin C, Brassinin derivatives, necrostatin 1/methylthiohydantoine-tryptophan (5-(1H-Indol-3-ylmethyl)-3-methyl-2-thioxo-4-Imidazolidinone 5-(Indol-3-ylmethyl)-3-methyl-2-thio-Hydantoin MTH-DL-Tryptophan), naphtoquinones, p-Coumarinic acid ((E)-3-(4-Hydroxyphenyl)-2-propenoic acid), Rosmarinic acid ((2R)-2-[[(2E)-3-(3,4-Dihydroxyphenyl)-1-oxo-2-propenyl]]oxy]-3-(3,4-dihydroxyphenyl)propanoic acid), and Epigallocatechin.

83. The pharmaceutical composition of claim 65, further comprising one or more anti-cancer agents.

84. The pharmaceutical composition of claim 83, wherein the anti-cancer agent is selected from the group consisting of cytotoxic T cells (CTL), cytotoxic antibodies, cytotoxic or growth-inhibitory chemotherapeutic agents, and anti-vasculature or anti-angiogenesis agents.

85.-88. (canceled)

89. A method for increasing proliferation of T lymphocytes, comprising
growing the T lymphocytes by culturing in vitro in the presence of an amount of one or more inhibitors of tryptophan 2,3-dioxygenase (TDO2) effective to increase the proliferation of the T lymphocytes at least about 10% relative to a control population of T lymphocytes that is cultured without the one or more TDO2 inhibitors.

90.-96. (canceled)


SEQUENCE LISTING (8 sequences)

SEQ ID NO: 1 - DNA, 23 bp, Artificial Sequence
  Oligonucleotide primer
ttggacttcaatgacttcagaga

SEQ ID NO: 2 - DNA, 17 bp, Artificial Sequence
  Oligonucleotide primer
tgcccagcattctgtgc

SEQ ID NO: 3 - DNA, 18 bp, Artificial Sequence
  Oligonucleotide primer
catggctggaaagaactc

SEQ ID NO: 4 - DNA, 19 bp, Artificial Sequence
  Oligonucleotide primer
ctgaagtgctctgtatgac

SEQ ID NO: 5 - DNA, 29 bp, Artificial Sequence
  Oligonucleotide primer
tttagagccacatggatttaacttctggg

SEQ ID NO: 6 - DNA, 19 bp, Artificial Sequence
  Oligonucleotide primer
ggcatcgtgatggactccg

SEQ ID NO: 7 - DNA, 19 bp, Artificial Sequence
  Oligonucleotide primer
gctggaaggtggacagcga

SEQ ID NO: 8 - DNA, 26 bp, Artificial Sequence
  Oligonucleotide primer
tcaagatcattgctcctcctgagcgc